(19) United States
(12) Patent Application Publication
(10) Pub. No.: US 2005/0075345 A1
(43) Pub. Date: Apr. 7, 2005

(54) Novel inhibitor compounds specific of secreted non-pancreatic human a<sb>2</sb>phospholipase of group II

(75) Inventors: Francoise Heymans, Pantin (FR); Aazdine Lamouri, Le Blanc Mesnil (FR); Jean-Jacques Godfroid, Paris (FR)

Correspondence Address: FULBRIGHT & JAWORSKI L.L.P., 600 CONGRESS AVE., SUITE 2400, AUSTIN, TX, 78701, US

(73) Assignee: Yang Ji Chemical Company Ltd., Anson, Kunggi-do (KR)

(21) Appl. No.: 10/497,742

(22) PCT Filed: Dec. 6, 2002
(86) PCT No.: PCT/FR02/04225

(30) Foreign Application Priority Data
Dec. 6, 2001 (FR) 01/15798

(51) Int. Cl. A61K 31/496; C07D 413/02

(52) U.S. Cl. 514/254.02; 544/369

(57) ABSTRACT

The present invention relates to a compound of the following formula (I) and pharmaceutical compositions containing the compound of formula (I): 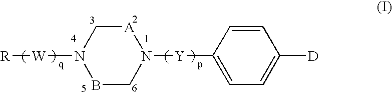 wherein D, Y, A, B, p, q, W and R have the same meanings as defined in the specification.

### BACKGROUND OF THE INVENTION

1. Field of the Invention

The present invention relates to novel specific inhibitor compounds of human non-pancreatic (group II) phospholipase A2 (hnps-PLA₂), their process of preparation, compositions containing the same, and their use particularly in the treatment of inflammatory pathologies.

2. Background of the Related Art

Subsequently to the penetration in pathogenic organisms (virus, bacteria, parasites or antigens) or in response to inflammatory stimulation (such as traumatism, burn or irradiation), PLA₂ play a pivot role in the propagation and amplification of inflammation. These enzymes catalyze the hydrolysis of phospholipid at the sn-2 position, and liberate fatty acid, such as arachidonic acid, and lysophospholipid. This fatty acid may act as a precursor to various lipids with a platelet-activating factor (PAF), leukotriene and prostaglandin. It causes multiple biological activities (e.g., cellular migration and proliferation, contraction, neurosecretion and hormone liberation, etc), and is involved in a variety of inflammatory pathologies and certain cancers.

Of phospholipases A₂ classified as EC 3.1.1.4 according to International Classification, the phospholipases A₂ of group II constitute a class of particular enzymes. The human non-pancreatic (group II) secretory phospholipase A2 (hnps-PLA₂) plays a central role, acting in an autocrine/paracrine manner, while participating in the production of proinflammatory lipid mediators and stimulating the cellular proliferation and migration through anti-bacterial properties. In various pathological situations, the ratio of circulating hnps-PLA₂ has a close correlation with the severity and issue of illness. This is the case in septic shock caused either by gram negative infection, peritonitis, malaria or even aspirin intoxication. In this case, the liberation of an excessive amount of hnps-PLA₂ contributes to circulatory collapse, hypotension, development of respiratory distress syndrome, and mortality. In rhumatoid arthritis, hnps-PLA₂ accumulates in cartilage, articular and extra-articular matrix, chondrocytes and synovial fluid, and the level of circulating enzyme is in accordance with the size and number of inflamed articulations. In the respiratory ways and lungs, hnps-PLA₂ is involved in asthma, allergic rhinitis, and asbestos. In the cardiovascular system, this enzyme is activated during ischemia (its expression is increased after cerebral ischemia shock) and plays an important role in high density lipoprotein deposition (there is a strong effect shown in the level of atheroma plaque), suggesting a potential role in atherosclerosis and cardiovascular morbidity. In the gastrointestinal tract, elevated concentrations of this enzyme are measured in Crohn's disease, ulcerative colitis and inflammatory bowel disease, as well as in cirrhosis and acute pancreatitis. In psoriasis, an increase in its activity is shown in skin lesions. In the brain, it could be involved in the cellar and tissue damages in cerebral ischemia and schizophrenia. Finally, it plays a role in plaque sclerosis and various cancers, particularly of the breast cancer and gastrointestinal tract.

The hnps-PLA₂ of group II is not the only secretory PLA₂ present in human organisms where the PLA₂ of groups I (pancreas), V (heart, lungs) and X (spleen, leukocyte, lungs) play an important role. The PLA₂ of groups V and X were recently discovered and their functions are poorly defined, and are involved in inflammation, like those of group II. However, the pancreatic secretory PLA₂ of group I have a primordial physiological role, because their catalytic activity is responsible for the digestion of lipids of alimentary origin. Thus, it is important that the enzymes have no influence on this function. This is complicated by the fact that all such enzymes have a similar size of 13 to 14 kDa, a three-dimensional structure (three alpha helices are connected by 6 to 8 disulfide bonds), and a dependence on calcium necessary for catalytic activity at concentrations of the order of mM. Furthermore, they possess the same mechanism of action based on a relay system of protons implicated in plural active site residues: histidine 48, glycine 30, aspartates 49 and 99 and tyrosines 52 and 73.

France Patent Application No. 99 06366 discloses compounds having an oxadiazolone type heterocycle, which can induce a very high selectivity on group II PLA₂ as compared to that on pancreatic (group I) PLA₂. This series of the compounds show high in vitro activity that reveals an in vivo activity similar to indomethacin (anti-inflammatory agent as reference compound), against the carrageenan-induced edema on the leg of rats when administered intraperitoneally. However, these compounds described in France Patent Application No. 99 06366 have low bioavailability when administered orally.

### SUMMARY OF THE INVENTION

The present invention provides a new class of group II PLA₂-selective inhibitor compounds, which have a superior inhibitory activity to that of compounds in the prior art, particularly the compounds described in France Patent Application No. 99 06366. The new compounds according to the present invention are particularly characterized by the presence of a substituted or unsubstituted piperazinyl ring on carbon atoms. The inventive compounds have a selective inhibitory activity on the PLA₂ of group II while they are completely inactive on the pancreatic PLA₂ of group I and also they possess a superior in vivo activity to that of indomethacin. Furthermore, the inventive compounds have an excellent bioavailability when administered orally.

An object of the present invention is compounds of the following formula (I): 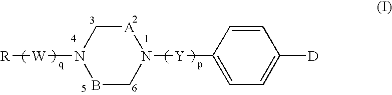
wherein:
D signifies a Z-HET group or a Z=HET group, and
(i) when D signifies the Z-HET group,
HET is a five-membered heterocycle, such as oxadiazolone of the following formula (II) or thiazolidine dione of the following formula (III): 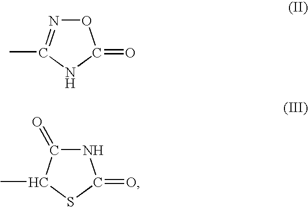
Z- is selected from the group consisting of —(CR₁R₂)ₙ— and —(CR₁═CR₂)ₙ— where n is an integer from 1 to 6, and R₁ and R₂, which may be the same or different, independently represents a hydrogen atom or a linear or branched alkyl group having 1 to 6 carbon atoms, and
(ii) when D signifies the Z=HET group,
Z- together with the heterocycle represent a -Z=HET group of the following formula (IV) or (V): 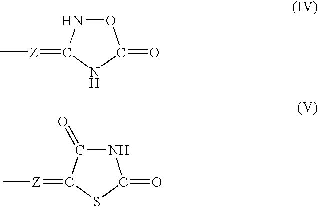
in which -Z= represents —CR₁═ where R₁ represents a hydrogen atom or a linear or branched alkyl group having 1 to 6 carbon atoms;
p is an integer of 0 or 1;
Y— is selected from the group consisting of C═O, SO₂ and —(CR₃R₄)ₘ— where m is an integer from 1 to 6, and R₃ and R₄, which may be the same or different, independently represents a hydrogen atom or a linear or branched alkyl group having 1 to 6 carbon atoms;
A and B on the piperazine cycle, which may be the same or different, independently represents either a carbon atom linked to hydrogen, or a carbon atom linked to both hydrogen and a linear or branched alkyl group having 1 to 3 carbon atoms, or a —C═O group;
q is an integer of 0 or 1;
W- is selected from the group consisting of 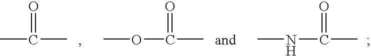
R is selected from the group consisting of a linear or branched alkyl group having 1 to 22 carbon atoms, a polyaryl group and aryl-alkyl, alkyl-Q-alkyl, alkyl-Q-aryl, aryl-Q-aryl, aryl-Q-aryl and aryl-Q-alkyl groups where “aryl” represents a substituted or unsubstituted 5- to 10-membered aryl group known to a person skilled in the art, particularly phenyl, naphthyl, phenylphenyl (or biphenyl) or heterocyclic aryl such as an indolyl group, with the aryl group being preferably substituted by at least one halogen atom, such as F, Cl or Br, or by a group selected from CF₃, OH, MeO and NO₂, “alkyl” represents a linear or branched alkyl group having 1 to 12 atoms, and “Q” is selected from the group consisting of —O—, —S—, —NH—, —NR₅—, —NH—CO—NH—, 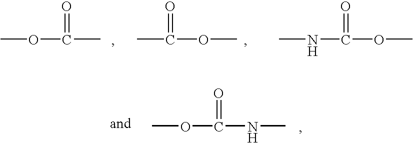
   where R₅ is a linear or branched alkyl group having 1 to 6 carbon atoms.

The group II PLA₂-selective inhibitory activity of the compounds of formula (I) defined above, which is expressed as the concentration of the compound (I) capable of inhibiting 50% of enzymatic activity (IC₅₀), is generally less than 1 μM, mainly less than 0.5 μM, and about 0.1 μM for certain compounds, whereas the most highly active compounds described in French Patent Application 99 06366 show an IC₅₀ of 3 μM.

A family of compounds according to the invention possessing a very high selective inhibitory activity against the human PLA₂ of group II is the family of compounds wherein p is 1, Y is a C═O group, and D, A, B, q, W and R have the meanings defined above.

According to a particularly advantageous embodiment of the present invention, the compounds of formula (I) are selected from the group consisting of the following compounds:
a) 1-[4′-(4,5-dihtdro-1,2,4(4H)-5-oxo-oxadiazol-3-ylmethyl)benzyl]-4-tetradecylpiperazine;
b) 1-[4′-(4,5-dihydro-1,2,4(4H)-5-oxo-oxadiazol-3-ylmethyl)benzoyl]-4-octadecylpiperazine;
c) 1-[4′-(4,5-dihydro-1,2,4(4H)-5-oxo-oxadiazole-3-ylmethyl)benzoyl]-2,5-dimethyl-4-dodecylpiperazine;
d) 1-[4′-(4,5-dihydro-1,2,4(4H)-5-oxo-oxadiazole-3-ylmethyl)phenyl]-4-octadecylpiperazine;
e) [4-(4′-octadecylpiperazine-1′-ylcarbonyl)benzylidene]-1,3-thiazolidine-2,4-dione;
f) 1-[4′-(2,4-dioxo-1,3-thiazolidine-5-ylmethyl)benzoyl]-4-octadecylpiperazine;
g) 1-[4′-(4,5-dihydro-1,2,4(4H)-5-oxo-oxadiazol-3-ylmethyl)benzyl]-4-tetradecylpiperazin-2-one;
h) 1-[4′-(4,5-dihydro-1,2,4(4H)-5-oxo-oxadiazol-3-ylethyl)benzoyl]-4-tetradecylpiperazine;
i) 1-[4′-(4,5-dihydro-1,2,4(4H)-5-oxo-oxadiazol-3-ylpropyl)benzoyl]-4-tetradecylpiperazine; and
j) 1-[4′-(4,5-dihydro-1,2,4(4H)-5-oxo-oxadiazol-3-yl-methylbenzoyl)-4-(N-octadecylaminocarbonyl)piperazine.

Another object of the present invention is to provide a process for the preparation of the compounds of formula (I). Generally, as illustrated by examples below, the inventive preparation process may be selected from the following processes (I) and (II):

Process (I) comprising the steps of: reacting hydroxylamine chlorohydrate with a derivative of the following formula (VI) to form the corresponding intermediate oxime; and subjecting the oxime obtained in the above step to cyclization reaction with chlorocarbonate (or chloroformate), followed by heating to a temperature sufficient to achieve a practically complete cyclization: 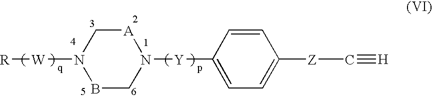
wherein R, W, A, B, p, q and Y have the same meanings as defined above; and Z is selected from the group consisting of —(CR₁R₂)ₙ— and —(CR₁═CR₂)ₙ— where n is an integer from 1 to 6, and R₁ and R₂, which may be the same or different, independently represents a hydrogen atom or a linear or branched alkyl group having 1 to 6 carbon atoms.

Process (II) comprising the step of: reacting thiazolidine-2,4-dione with the aldehyde functional group of a derivative of the following formula (VII): 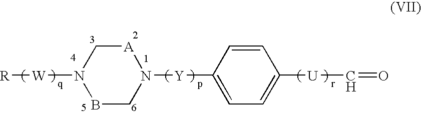
wherein R, W, A, B, p, q and Y have the same meanings as defined above; r is an integer of 0 or 1; U is selected from the group consisting of —(CR₆R₇)ₛ— and —(CR₆═CR₇)ₛ— where s is an integer from 1 to 6, and R₆ and R₇ which may be the same or different each independently represents an hydrogen atom or a linear or branched alkyl group having 1 to 6 carbon atoms. This reaction is carried out under reflux in toluene in the presence of pyridinium benzoate to form the ethylene derivative (V) as described above, and then optionally, reduction of the double linkage Z=C is performed by catalytic hydrogenation (Parr apparatus) in the presence of 100% palladium black and hydrogen under pressure in absolute ethanol at 50° C.

The starting materials used in the above steps can be prepared by any method known to a person in the art (particularly, method of Examples 1-6 below).

Furthermore, the present invention concerns the use of the compound of formula (I) for the selective inhibition of group II PLA₂ in an in vitro test.

Still another object of the present invention is to provide a pharmaceutical composition comprising at least one compound of formula (I) in combination with at least one excipient selected from the group consisting of pharmaceutically acceptable excipients.

For formulation of the pharmaceutical composition according to the present invention, a person skilled in the art may advantageously make reference to the most recent edition of the United States Pharmacopeia. Particularly, a person skilled in the art may advantageously make reference to the fourth edition (2002) of European Pharmacopeia and the USP 25-NF20 edition of the United States Pharmacopeia.

Advantageously, the inventive pharmaceutical composition as defined above is adapted for oral or parenteral administration at the amount of a formula (I) compound of 1 μg-10 mg, and preferably 1 μg-1 mg, per kg of the body weight of a patient.

Advantageously, the inventive pharmaceutical composition as defined above is adapted for local or topical administration at the amount of a formula (I) compound of 1 μg-100 mg, and preferably 100 μg-10 mg, per kg of the body weight of a patient.

If the inventive composition comprises at least one pharmaceutically acceptable excipient, this excipient may be an excipient suitable for topical administration of the composition, an excipient suitable for oral administration of the composition and/or an excipient suitable for parenteral administration of the composition.

Finally, with reference to a biological activity assay below, still another object of the present invention is to provide the use of the compound of formula (I) as a therapeutic active ingredient in a medicament.

Particularly, the present invention provides the use at least one compound of formula (I) for the preparation of a medical composition for inhibiting the activity of human non-pancreatic (group II) secretory PLA₂.

Furthermore, the present invention provides the use of at least one compound of formula (I) for the preparation of a medicament for preventing or treating chronic and acute inflammations, particularly inflammatory pathologies related to non-pancreatic secretory PLA₂.

Examples of inflammatory pathologies as mentioned above include rheumatoid polyarthritis, septic shock, infection, peritonitis, malaria or even aspirin intoxication, circulatory collapse, hypotension, respiratory distress syndrome, asthma, allergic rhinitis, acute lung injury, asbestos, anemia, atherosclerosis, cardiovascular morbidity, Crohn's disease, ulcerative colitis, inflammatory bowel disease, as well as in cirrhosis, acute pancreatitis, psoriasis, the cellular and tissue lesions in cerebral ischemia and schizophrenia, and other pathological diseases, for example, plaque sclerosis and certain cancers of the breast and gastrointestinal tract.

Moreover, the present invention provides the use of at least one compound of formula (I) for the preparation of a medicament for treating rheumatic troubles.

Also, the present invention provides a method for treating inflammatory conditions in a patient, and preferably acute or chronic inflammatory conditions, the method comprising the step of administering to the patient a therapeutic effective amount of the compound of formula (I), or the pharmaceutical composition containing the compound of formula (I).

In addition, the present invention provides a method for preventing inflammatory conditions in a patient, the method comprising the step of administering to the patient a therapeutic effective amount of the compound of formula (I), or the pharmaceutical composition containing the compound of formula (I).

The compound of formula (I), or the pharmaceutical composition containing the compound of formula (I), may be administered by an oral or parenteral route or applied topically or locally on the skin of a patient.

### DETAILED DESCRIPTION OF THE PREFERRED EMBODIMENT

The present invention will hereinafter be described in further detail by examples. It should however be borne in mind that the present invention is not limited to or by the examples.

### EXAMPLE 1

### Preparation of 1-[4′-(4,5-dihydro-1,2,4(4H)-5-oxo-oxadiazol-3-ylmethyl)benzyl]-4-tetradecylpiperazine

(Compound of formula wherein D=Z-HET, HET=oxadiazolone (II), Z=—CH₂—, n=1, Y=—CH₂—, A=B=—CH₂—, and R=—(CH₂)₁₃—CH₁₃)

### 1-1: Preparation of Tetradecylpiperazine

In a 250 ml erlenmeyer flask, 13 g (0.151 mol) of piperazine dissolved in 100 ml of a mixture of THF/CH₂Cl₂ (3:1 v/v) was stirred. 4.24 g (15 mmol) of 1-bromotetradecane was added to the mixture, followed by stirring for one hour at ambient temperature. Then, the solvent was evaporated, and the resulting residue was taken up in dichloromethane, and washed two times with water. The organic phase was dried over MgSO₄, filtered and evaporated. After crystallization from an acetone/ether mixture at −18° C., 3.4 g of white crystal was obtained, which melts at ambient temperature. Yield: 80%. Rf: 0.40 (CH₂Cl₂/MeOH/NH₄OH, 80:20:2 v/v/v).

IR (KBr): 3440 (N—H) cm⁻¹

¹H NMR (200 MHz, CDCl₃, HMDS) δ ppm: 6-8 (most, 1H, NH), 2.85 and 2.33 (2t, 8H, J=4.88 and 4.50 Hz, H of piperazine), 2.21 (t, 2H, J=7.56 Hz, CH₂—N), 1.40 (m, 2H, CH₂—C—N), 1.20 (s1, 22H, CH₂), 0.80 (t, 3H, J=6.62 Hz, CH₃).

### 1-2: Preparation of 1-(4′-cyanomethylbenzyl)-4-tetradecylpiperazine

### a) 4-Preparation of Bromomethylphenyl Acetonitrile

In a 1-liter Erlenmeyer flask, 25 g (0.19 mol) of 4-methylphenyl acetonitrile was dissolved in 300 ml of carbon tetrachloride. To the solution, 41 g (0.23 mol) of N-bromosuccinimide (NBS) and 0.5 g of 2,2′-azobis(2-methylpropionitrile) (AIBN) which had been crystallized in acetic acid were added. The resulting solution was heated under reflux for 3 hours. At the end of the reaction, the solution was cooled and then washed three times with water. The organic phase was dried over MgSO₄, filtered and evaporated under vacuum. Distillation of the residue under reduced pressure (1 mmHg) allowed successive recovery of three fractions at 95° C., 110° C. and 140° C. The final fraction at 140° C., which corresponds to the desired compound 4-bromomethylphenyl acetonitrile, was crystallized from ether at −18° C., to produce 14 g of white crystal. Yield: 35%. Melting point: 63° C. Rf: 0.19 (ether/petroleum ether, 30:70 v/v).

IR(KBr): 2224 (C≡N), 1594 (C═Cₐᵣ) cm⁻¹

¹H NMR (200 MHz, CDCl₃, HMDS) δ ppm: 7.34 and 7.24 (2d, 4H, J=8.30 and 8.27 Hz, aromatic H), 4.41 (s, 2H CH₂—Br), 3.68 (s, 2H, CH₂—C≡N).

### b) Preparation of 1-(4′-cyanomethylbenzyl)-4-tetradecylpiperazine

In a 250 ml Erlenmeyer flask equipped with a cooler and a calcium chloride guard, 7 g (24 mmol) of 1-tetradecylpiperazine, 6 g (28 mmol) of 4-bromomethylphenyl acetonitrile, 9.93 g (71 mmol) of potassium carbonate and 0.5 g of potassium iodide were mixed with each other in 200 ml of acetonitrile. The mixture was heated under reflux for 6 hours. At the end of the reaction, the suspension was filtered, and K₂CO₃ was rinsed out several times with dichloromethane. The solvent was evaporated under vacuum, and the residue was taken up in 150 ml of dichloromethane and washed with water until neutral pH. The organic phase was dried over MgSO₄, filtered, and concentrated under reduced pressure. The residue was purified by chromatography on a silica gel column using a MeOH/CH₂Cl₂ mixture (1:99 v/v) as eluent, to produce 8.2 g of the title nitrile as brown oil. Yield: 83%. Rf: 0.33 (CH₂Cl₂/MeOH, 95:5 v/v).

IR (KBr): 2248 (C≡N), 1607 (C═Cₐᵣ) cm⁻¹

¹H NMR (200 MHz, CDCl₃, HMDS) δ ppm: 7.26 and 7.18 (2d, 4H, J=8.09 and 9.52 Hz, aromatic H), 3.64 (s, 2H, CH₂—C≡N), 3.42 (s, 2H, Ph-CH₂—N), 2.40 (m, 8H, piperazine H), 2.25 (t, 2H, J=7.64 Hz, CH₂—N), 1.39 (m, 2H, CH₂—C—N), 1.18 (s1, 22H, CH₂), 0.80 (t, 3H, J=6.13 Hz, CH₃).

### 1-3: Preparation of 1-[4′-(N-hydroxyamidinomethyl)benzyl]-4-tetradecylpiperazine

In a 250 ml erlenmeyer flask equipped with an addition ampoule and a cooler, 13.08 g (94 mmol) of potassium carbonate, and 5.48 g (78 mmol) of hydroxylamine chlorohydrate were suspended in 150 ml of absolute ethanol, and the mixture was heated under reflux. To the suspension, 6.5 g (15 mmol) of 1-(4′-cyanomethylbenzyl)-4-tetradecylpiperazine in 150 ml of anhydrous ethanol was added dropwise. The reaction mixture was stirred under reflux for 24 hours. At the end of the reaction, the salt was filtered at low temperature and washed several times with dichloromethane. The filtrate was concentrated under reduced pressure, and taken up in dichloromethane. The organic phase was washed until neutralization, dried over MgSO₄ and filtered. After evaporating the solvent, the residue was crystallized from acetone, to produce 4.62 g of the title amidoxime as white crystal. Yield: 65%. Melting point: 74° C. Rf: 0.28 (CH₂Cl₂/MeOH, 90:10 v/v).

IR (KBr): 3490 (O—H), 3374 (NH₂), 1655 (C═N), 1607 (C═Cₐᵣ) cm⁻¹

¹H NMR (200 MHz, CDCl₃, HMDS) δ ppm: 7.20 and 7.14 (2d, 4H, J=7.39 and 8.10 Hz, aromatic H), 4.41 (s, 2H, NH₂), 3.41 (s, 2H, CH₂—C═N), 3.35 (s, 2H, Ph-CH₂—N), 2.41 (m, 8H, piperazine H), 2.25 (t, 2H, J=7.64 Hz, CH₂—N), 1.36 (m, 2H, CH₂—C—N), 1.18 (s1, 22H, CH₂), 0.80 (t, 3H, J=6.13 Hz, CH₃).

### 1-4: Preparation of 1-[4′-(4,5-dihydro-1,2,4(4H)-5-oxooxadiazol-3-ylmethyl)benzyl]-4-tetradecylpiperazine

This synthesis was carried out in two steps as described below. In a 100 ml round-bottomed flask, 1.8 g (4 mmol) of amidoxime and 0.66 ml of (4 mmol) of triethylamine were dissolved in 40 ml of anhydrous dichloromethane. The solution was stirred at 0° C. for one hour, after which 0.60 ml (5 mmol) of phenyl chloroformate was added to the reaction mixture. After stirring at 0° C. for one hour, the solution was washed with alkaline solution (saturated Na₂CO₃), washed three times with water, dried over MgSO₄, filtered, and then concentrated under vacuum. The carbonate intermediate obtained was taken in 40 ml of anhydrous toluene, and heated under reflux for 12 hours. The toluene was evaporated under reduced pressure, and the resulting residue was purified by chromatography on a silica gel column using a CH₂Cl₂/MeOH mixture (98:2 v/v) as eluent. The crude product was crystallized from an acetone/ether mixture, to produce 500 mg of the final compound as white crystal.

Yield: 26%. Melting point: 98° C. Rf: 0.33 (CH₂Cl₂/MeOH, 90:10 v/v).

IR (KBr): 1732 (NC═O), 1688 (C═N), 1599 (C═Cₐᵣ) cm⁻¹

¹H NMR (200 MHz, CDCl₃, HMDS) δ ppm: 8-12 (most, 1H, NH), 7.27 and 7.18 (2d, 4H, 7=8.07 and 9.12 Hz, aromatic H), 3.67(s, 2H, CH₂—C═N), 3.35 (s, 2H, Ph-CH₂—N), 2.56 and 2.34 (2m, 8H, piperazine H), 2.46 (t, 2H, J=7.64 Hz, CH₂—N), 1.47 (m, 2H, CH₂—C—N), 1.18 (s1, 22H, CH₂), 0.80 (t, 3H, J=6.13 Hz, CH₃).

### EXAMPLE 2

### 1-[4′-(4,5-dihydro-1,2,4(4H)-5-oxo-oxadiazol-3-ylmethyl)benzoyl]-4-octadecylpiperazine

(Compound of formula (I) wherein D=Z-HET, HET=oxadiazolone of formula (II), Z=—CH₂—, n=1, Y=C═O, A=B=—CH₂—, and R=—(CH₂)₁₇—CH₃)

### 2-1: Preparation of 1-octadecylpiperazine

The same procedure as described in the step 1-1 of Example 1 was repeated except that 13 g (0.151 mol) of piperazine and 5 g (15 mmol) of 1-bromooctadecane were used as starting materials, and 4.5 g of white crystal was obtained after crystallization from acetone.

Yield: 89%. Melting point: 61.5° C. Rf: 0.40 (CH₂Cl₂/MeOH/NH₄OH, 80:20:2 v/v/v).

IR (KBr): 3440 (N—H) cm⁻¹

¹H NMR (200 MHz, CDCl₃, HMDS) δ ppm: 6-8 (s1, 1H, NH), 2.85 and 2.33 (2t, 8H, J=4.88 and 4.50 Hz, piperazine H), 2.21 (t, 2H, J=7.56 Hz, CH₂—N), 1.40 (m, 2H, CH₂—C—N), 1.20 (s1, 30H, CH₂), 0.80 (t, 3H, J=6.62 Hz, CH₃).

### 2-2: Preparation of 4-bromomethylbenzoyl Chloride

In a 250 ml round-bottomed flask equipped with a cooler and a calcium chloride guard, 8.4 g (54 mmol) of 4-methylbenzoyl chloride and 9.66 g (54 mmol) of N-bromosuccinimide (NBS) previously crystallized in acetic acid, and 0.5 g of 2,2′-azobis(2-methylpropinonitrile) (AIBN) in 150 ml of carbon tetrachloride, were dissolved. The solution was heated under reflux for three hours. At the end of the reaction, the salt was filtered, and the solution was cooled again and then washed three times with water. The organic phase was dried over MgSO₄, filtered, and concentrated under vacuum. The residue was crystallized from pantane, to produce 9 g of white crystal. Yield: 72%. Melting point: 86.7° C.

¹H NMR (200 MHz, CDCl₃, HMDS) δ ppm: 8.02 and 7.46 (2d, 4H, J=8.38 and 8.29 Hz, aromatic H), 4.43 (s, 2H, CH₂—Br).

### 2-3: Preparation of 1-(4′-bromomethylbenzoyl)-4-octadecylpiperazine

In a 250 ml erlenmeyer flask equipped with an addition ampoule and a calcium chloride guard, 4.4 g (13 mmol) of octadecylpiperazine and 2.7 ml (19 mmol) of triethylamine were dissolved in 100 ml of anhydrous benzene. The mixture was stirred at 0° C., to which 3.04 g (13 mmol) of 4-bromomethylbenzoyl chloride was then added dropwise. After stirring for two hours at ambient temperature, the solvent was evaporated, and the resulting residue was taken up in dichloromethane. The solution was washed with alkaline solution, and then washed several times with water until neutralization. The organic phase was dried over MgSO₄, filtered, and concentrated under vacuum. The crude product was purified by chromatography on a silica gel column with dichloromethane as eluent. This yielded 5 g of a pure product as oil. Yield: 72%. Rf: 0.50 (CH₂Cl₂/MeOH, 95:5 v/v).

IR (KBr): 1624 (NC═O), 1607 (C═Cₐᵣ) cm⁻¹

¹H NMR (200 MHz, CDCl₃, HMDS) δ ppm: 7.34 (s, 4H, aromatic H), 4.52 (s, 2H, CH₂—Br), 3.72 and 3.38 (2m, 4H, CH₂—N—C═O of piperazine), 2.43 (m, 4H, H of piperazine), 2.33 (t, 2H, J=7.65 Hz, CH₂—N), 1.41 (m, 2H, CH₂—CN), 1.18 (s1, 30H, CH₂), 0.80 (t, 3H, J=6.13 Hz, CH₃).

### 2-4: Preparation of 1-(4′-cyanomethylbenzoyl)-4-octadecylpiperazine

In a 250 ml Erlenmeyer flask equipped with a cooler and a calcium chloride guard, 5.35 g (10 mmol) of the bromide derivative prepared in the above step 2-3 was dissolved in 70 ml of dimethylsulfoxide. The solution was stirred at 0° C., to which 1.96 g (40 mmol) of sodium cyanide was then added in portions. The mixture was brought to ambient temperature and then heated at 80° C. for one hour. The reaction mixture was diluted with dichloromethane and water. The organic phase was washed several times with water, dried over MgSO₄, filtered, and concentrated under vacuum. The residue was purified by chromatography on a silica gel column with dichloromethane as eluent. This yielded 3 g of the title nitrile as thick honey-colored oil. Yield: 61%. Rf: 0.5(CH₂Cl₂/MeOH, 97:3 v/v).

IR (KBr): 2251 (C≡N), 1620 (NC═O), 1607 (C═Cₐᵣ) cm⁻¹

¹H NMR (200 MHz, CDCl₃, HMDS) δ ppm: 7.36 and 7.30 (2d, 4H, J=8.51 and 8.48 Hz, aromatic H), 3.71 (s, 2H, CH₂—C≡N), 3.72 and 3.38 (2m, 4H, CH₂—N—C═O of piperazine), 2.43 (m, 4H, H of piperazine), 2.33 (t, 2H, J=7.65 Hz, CH₂—N), 1.41 (m, 2H, CH₂—C—N), 1.18 (s1, 30H, CH₂), 0.81(t, 3H, J=6.13 Hz, CH₃).

### 2-5: Preparation of 1-[4′-(N-hydroxyamidinomethyl)benzoyl]-4-octadecyl Piperazine

The same procedure as described in the step 1-3 of Example 1 was performed except that 6 g (12 mmol) of 1-(4′-cyanomethylbenzoyl)-4-octadecylpiperazine, 10.26 g (74 mmol) of potassium carbonate and 4.30 g (61 mmol) of hydroxylamine chlorohydrate were used. The crude product was crystallized from acetone to produce 4 g of the title oxime as white crystal. Yield: 67%. Melting point: 105.2° C. Rf: 0.39 (CH₂Cl₂/MeOH, 90:10 v/v).

IR (KBr): 3486 (O—H), 3373 (NH₂), 1657 (NC═O), 1625 (C═N), 1582 (C═Cₐᵣ) cm⁻¹

¹H NMR (200 MHz, CDCl₃, HMDS) δ ppm: 7.30 and 7.24 (2d, 4H, J=8.28 and 8.11 Hz, aromatic H), 4.43 (s, 2H, NH₂), 3.42 (s, 2H, CH₂—C═N), 3.73 and 3.40 (2m, 4H, CH₂—N—C═O of piperazine), 2.55 (m, 4H, H of piperazine), 2.29 (t, 2H, J=6.64 Hz, CH₂—N), 1.39 (m, 2H, CH₂—C—N), 1.18 (s1, 30H, CH₂), 0.81 (t, 3H, J=6.03 Hz, CH₃).

### 2-6: Preparation of 1-[4′-(4,5-dihydro-1,2,4(4H)-5-oxooxadiazol-3-ylmethyl)benzoyl]-4-octadecylpiperazine

The same procedure as described in the step 1-4 of Example 1 was performed except that 1.3 g (2.53 mmol) of the amidoxime prepared in the above step 2-5, 0.45 ml (3.28 mmol) of triethylamine and 0.4 ml (3.03 mmol) of phenyl chloroformate were used. The product was crystallized from acetone to produce 500 mg of the final compound as white crystals. Yield: 37%. Melting point: 121° C. Rf: 0.38 (CH₂Cl₂/MeOH, 90:10 v/v).

IR (KBr): 1780 (OC═O), 1734 (C═N), 1640 (NC═O), 1607 (C═Cₐᵣ) cm⁻¹

¹H NMR (200 MHz, CDCl₃, HMDS) δ ppm: 8-12 (most, 1H, NH), 7.16 (s, 4H, aromatic H), 3.79 (s, 2H, CH₂—C═N), 3.77 and 3.36 (2m, 4H, CH₂—N—C═O of piperazine), 2.52 (m, 4H, H of piperazine), 2.35 (t, 2H, J=5.86 Hz, CH₂—N), 1.43 (m, 2H, CH₂—C—N), 1.18 (s1, 30H, CH₂), 0.81 (t, 3H, J=6.21 Hz, CH₃).

### EXAMPLE 3

### Preparation of 1-[4′-(4,5-dihydro-1,2,4(4H)-5-oxo-oxadiazol-3-ylmethyl)benzoyl]-2,5-dimethyl-4-dodecylpiperazine

(Compound of formula (I) wherein D=Z-HET, HET=oxadiazolone of formula (II), Z=—CH₂—, n=1, Y=C═O, A=B=CH—CH₃, and R=—(CH₂)₁₁—CH₃)

### 3-1: Preparation of 2,5-dimethyl-1-dodecylpiperazine

The same procedure as described in the step 1-1 of Example 1 was performed except that 3.27 g (13 mmol) of bromododecane and 12 g (0.105 mol) of trans-2,5-dimethylpiperazine in 170 ml THF were used as starting materials. This yielded 2.8 g of the title substituted piperazine as oil. Yield: 76%. Rf: 0.3(CH₂Cl₂/MeOH/NH₄OH, 80:20:2 v/v/v).

IR (KBr): 3440 (N—H) cm⁻¹

¹H NMR (200 MHz, CDCl₃, HMDS) δ ppm: 6-8 (most, 1H, NH), 2.29 (m, 8H, CH₂—N and H of piperazine), 1.36 (m, 5H, CH₃ on piperazine and CH₂C—N), 1.19 (s1, 18H, CH₂ on piperazine), 0.98 (s1, 3H, CH₃), 0.81 (t, 3H, J=6.73 Hz, CH₃).

### 3-2: Preparation of 1-(4′-chloromethylbenzoyl)-2,5-dimethyl-4-dodecylpiperazine

In a 250 ml Erlenmeyer flask, 6 g (21 mmol) of the substituted piperazine prepared in the above step 3-1 and 3.18 g (31 mmol) of triethylamine were dissolved in 150 ml of benzene. The mixture was stirred at 0° C., to which 4.82 g (25 mmol) of 4-chloromethylbenzoyl chloride (commercial or prepared in the same manner as in the step 2-2 of Example 2 except for the use of N-chlorosuccinimide) was then added dropwise. After stirring for 3 hours at ambient temperature, benzene was evaporated, and the residue was taken up in dichloromethane, washed with Na₂CO₃ saturated solution, and then washed two times with water. The organic phase was dried over MgSO₄, filtered, and concentrated under vacuum. The residue was purified by chromatography on a silica gel column with dichloromethane as eluent. This yielded 4.33 g of the chloride derivative as oil. Yield: 48%. Rf: 0.36 (CH₂Cl₂/MeOH, 95:5 v/v).

IR (KBr): 1624 (NC═O), 1595 (C═Cₐᵣ) cm⁻¹

¹H NMR (200 MHz, CDCl₃, HMDS) δ ppm: 7.34 and 7.27 (2d, 4H, J=8.40 and 7.96 Hz, aromatic H), 4.52 (s, 2H, CH₂—CI), 3.36 and 2.62 (2d, 4H, J=7.40 and 7.67 Hz, CH₂ of piperazine), 2.88 and 2.28 (2m, 2H, CH of piperazine), 2.24 (t, 2H, J=5.52 Hz, CH₂—N), 1.40 (m, 2H, CH₂—C—N), 1.35 and 0.94 (2d, 6H, CH₃ of piperazine), 1.18 (s1, 18H, CH₂), 0.81 (t, 3H, J=6.74 Hz, CH₃).

### 3-3: Preparation of 1-(4′-cyanomethylbenzoyl)-2,5-dimethyl-4-dodecylpiperazine

4.33 g (9.96 mmol) of the chloride derivative prepared in the above step 3-2 was dissolved in 50 ml of DMSO. To the solution which had been stirred at 0° C., 1.49 g (29 mmol) of sodium cyanide was added in small portions. After completion of the addition, the solution was heated at 80° C. for one hour. At the end of the reaction, extraction was performed with the addition of a mixture of dichloromethane and water. The organic phase was washed two times with water, dried over MgSO₄, filtered, and concentrated under vacuum. The resulting residue was purified by chromatography on a silica gel column using dichloromethane as eluent. This yielded 4 g of the title pure nitrile as oil. Yield: 94%. Rf: 0.5 (CH₂Cl₂/MeOH, 95:5 v/v)

IR (KBr): 2245 (C≡N), 1611 (NC═O), 1607 (C═Cₐᵣ) cm⁻¹

¹H NMR (200 MHz, CDCl₃, HMDS) δ ppm 7.30 (s, 4H, aromatic H), 3.71 (s, 2H, CH₂—C≡N), 3.36 and 2.62 (2d, 4H, J=7.40 and 7.67 Hz, CH₂ of piperazine), 2.88 and 2.28 (2m, 2H, CH of piperazine), 2.24 (t, 2H, J=5.52 Hz, CH₂—N), 1.40 (m, 2H, CH₂—C—N), 1.28 and 0.84 (2d, 6H, CH₃ of piperazine), 1.18 (s1, 18H, CH₂), 0.81 (t, 3H, J=6.74 Hz, CH₃).

### 3-4: Preparation of 1-[4′-(N-hydroxyamidinomethyl)benzoyl]-2,5-dimethyl-4-dodecylpiperazine

The same procedure as described in the step 1-3 of Example 1 was repeated except that 4 g (9.41 mmol) of the nitrile prepared in the above step 3-3, 3.26 g (47 mmol) of hydroxylamine chlorohydrate and 7.79 g (56 mmol) of potassium bicarbonate were used. After purification, 1.6 g of amidoxime as oil was obtained. Yield: 37%. Rf: 0.46 (CH₂Cl₂/MeOH, 90:10 v/v).

IR (KBr): 3369 (O—H), 3328 (NH₂), 1661 (NC═O), 1612 (C═N), 1595 (C═Cₐᵣ) cm⁻¹

¹H NMR (200 MHz, CDCl₃, HMDS) δ ppm: 7.30 (s, 4H, aromatic H), 3.36 and 2.62 (2d, 4H, J=7.40 and 7.67 Hz, CH₂ of piperazine), 5.70 (s1, 1H, OH), 4.44 (s1, 2H, NH₂), 3.39 (s, 2H, CH₂—C═N), 2.88 and 2.28 (2m, 2H, CH of piperazine), 2.24 (t, 2H, J=5.52 Hz, CH₂—N), 1.40 (m, 2H, CH₂—C—N), 1.28 and 0.84 (2d, 6H, CH₃ on piperazine), 1.18 (s1, 18H, CH₂), 0.81 (t, 3H, J=6.74 Hz, CH₃).

### 3-5: Preparation of 1-[4′-(4,5-dihydro-1,2,4 (4H)-5-oxo-oxadiazol-3-ylmethyl)benzoyl]-2,5-dimethyl-4-dodecylpiperazine

The same procedure as described in the step 1-4 of Example 1 was repeated except that 1.6 g (3.49 mmol) of the amidoxime prepared in the above step 3-4, 0.58 ml (4.19 mmol) of triethylamine and 0.48 ml (3.83 mmol) of phenyl chloroformate were used as starting materials. The resulting residue was purified by chromatography on a silica gel column with dichloromethane as eluent. This produced 600 mg of the final pure compound as foams. Yield: 35%. Rf: 0.4 (CH₂Cl₂/MeOH, 90:10 v/v).

IR (KBr): 1670 (NOC═O), 1634 (C═N), 1595 (C═Cₐᵣ) cm⁻¹

¹H NMR (200 MHz, CDCl₃, HMDS) δ ppm: 7.14 (s, 4H, aromatic H), 6.11 (s1, 1H, NH), 3.74 (s, 2H, CH₂—C═N), 3.36 and 2.62 (2d, 4H, J=7.40 and 7.67 Hz, CH₂ of piperazine), 2.88 and 2.28 (2m, 2H, CH of piperazine), 2.24 (t, 2H, J=5.52 Hz, CH₂—N), 1.40 (m, 2H, CH₂—C—N), 1.28 and 0.84 (2d, 6H, CH₃ on piperazine), 1.18 (s1, 18H, CH₂), 0.81 (t, 3H, J=6.74 Hz, CH₃).

### EXAMPLE 4

### Preparation of 1-[4′-(4,5-dihydro-1,2,4(4H)-5-oxo-oxadiazol-3-ylmethyl)phenyl]-4-octadecylpiperazine

(Compound of formula (I) wherein D=Z-HET, HET=oxadiazolone of formula (II), Z=—CH₂—, n=1, p=0, A=B=—CH₂—, and R=—(CH₂)₁₇—CH₃)

### 4-1: Preparation of N-octadecyldiethanolamine

In a 500 ml Erlenmeyer flask, 10 g (95 mmol) of diethanolamine, 37.96 g (0.114 mol) of octadecane bromide, 39.33 g (0.285 mol) of potassium bicarbonate and 0.5 g of potassium iodide in 200 ml of acetonitrile were mixed. The reaction mixture was stirred and heated under reflux for 3 hours. At the end of the reaction, the solvent was evaporated and the residue was taken up in dichloromethane. The organic phase was washed two times with water, dried over MgSO₄, filtered, and concentrated under vacuum. The residue was crystallized from acetone, to obtain 33 g of white crystal. Yield: quantitative. Melting point: 49° C. Rf: 0.20 (CH₂Cl₂/MeOH, 90:10 v/v).

IR (KBr): 3310 (O—H) cm⁻¹

¹H NMR (200 MHz, CDCl₃, HMDS) δ ppm: 3.53 (t, 4H, J=5.43 Hz, CH₂—O), 3.27 (s1, 2H, OH), 2.57 (t, 4H, J=5.43 Hz, N—CH₂—C—O), 2.44 (t, 2H, J=7.06 Hz, CH₂—N), 1.34 (m, 2H, CH₂—C—N), 1.18 (s1, 30H, CH₂), 0.80 (t, 3H, J=5.85 Hz, CH₃)

### 4-2: Preparation of N,N′-di(chloroethyl)octadecylamine

In a 250 ml Erlenmeyer flask, 13 g (36 mmol) of N-octadecylamine in 100 ml of chloroform was dissolved and cooled to 0° C. Then, 7.95 ml (0.109 mol) of thionyl chloride was added dropwise to the cooled material. After completion of the addition, the reaction mixture was heated under chloroform reflux for 3 hours. An excess of the solvent and thionyl chloride were evaporated, and the residue taken up in dichloromethane was washed with Na₂CO₃ saturated solution, and washed several times with water until neutralization. The organic phase was dried over MgSO₄, filtered, and concentrated under vacuum. The residue was purified by chromatography on a silica gel column using an ether/petroleum ether mixture (5:95 v/v) as eluent. This yielded 10 g of pure amine chloride as oil. Yield: 70%. Rf: 0.43 (ether/petroleum ether, 5:95 v/v).

¹H NMR (200 MHz, CDCl₃, HMDS) δ ppm: 3.38 (t, 4H, J=5.43 Hz, CH₂—Cl), 2.75 (t, 4H, J=7.30 Hz, N—CH₂—C—Cl), 2.43 (t, 2H, J=6.67 Hz, CH₂—N), 1.36 (m, 2H, CH₂—C—N), 1.16 (s1, 30H, CH₂), 0.80 (t, 3H, J=5.85 Hz, CH₃).

### 4-3: Preparation of 1-(4′-cyanomethylphenyl)-4-octadecylpiperazine

In a 250 ml round-bottomed flask, 3 g (7.6 mmol) of N,N′-di(chloroethyl)octadecylamine, 2 g (15 mmol) of 4-aminophenylacetonitrile and 0.2 g of potassium iodide in 100 ml of acetonitrile were mixed. The suspension was stirred under reflux for 16 hours. At the end of the reaction, the solvent was evaporated, and the residue was taken up in dichloromethane, washed with basic solution, and then washed several times with water. The organic phase was dried over MgSO₄, filtered, and concentrated under vacuum. The resulting residue was crystallized from acetone, to produce 2.66 g of the title piperazine as white crystal. Yield: 77%. Melting point: 94° C. Rf: 0.33 (CH₂Cl₂/MeOH, 98:2 v/v).

IR (KBr): 2252 (C≡N), 1607 (C═Cₐᵣ) cm⁻¹

¹H NMR (200 MHz, CDCl₃, HMDS) δ ppm: 7.17 and 6.84 (2d, 4H, J=8.60 and 8.63 Hz, aromatic H), 3.62 (s, 2H, CH₂—C≡N), 3.61 and 3.22 (2s1, 8H, H of piperazine), 2.92 (t, 2H, J=8.32 Hz, CH₂—N), 1.85 (m, 2H, CH₂—C—N), 1.19 (s1, 30H, CH₂), 0.81 (t, 3H, J=5.90 Hz, CH₃).

### 4-4: Preparation of 1-[4′-(N-hydroxyamidinomethyl)phenyl]-4-octadecylpiperazine

The same procedure as described in the step 1-3 of Example 1 was repeated except that 1.52 g (21 mmol) of hydroxylamine chlorohydrate, 3.64 g (26 mmol) of potassium carbonate and 2 g of the nitrile prepared in the above step 4-3 were used. The crude product was purified by chromatography on a silica gel column using dichloromethane as eluent, and the resulting oil was crystallized from acetone to produce 600 mg of the title amidoxime as white crystal. Yield: 28%. Melting point: 110.1° C. Rf: 0.40 (CH₂Cl₂/MeOH, 95:5 v/v).

IR (KBr): 3489 (O—H), 3375 (NH₂), 1655 (C═N), 1607 (C═Cₐᵣ) cm⁻¹

¹H NMR (200 MHz, CDCl₃, HMDS) δ ppm: 7.08 and 6.80 (2d, 4H, J=8.60 and 8.62 Hz, aromatic H), 4.36 (s, 2H, NH₂), 3.44 (s, 2H, CH₂—C═N), 3.14 and 2.56 (2s1, 8H, H of piperazine), 2.34 (t, 2H, J=7.34 Hz, CH₂—N), 1.47 (m, 2H, CH₂—C—N), 1.19 (s1, 30H, CH₂), 0.81 (t, 3H, J=6.05 Hz, CH₃).

### 4-5: Preparation of 1-[4′-(4,5-dihydro-1,2,4(4H)-5-oxo-oxadiazol-3-ylmethyl)phenyl]-4-octadecylpiperazine

The same procedure as described in the step 1-4 of Example 1 was repeated except that 600 mg (1.2 mmol) of the amidoxime prepared in the above step 4-4, 0.22 ml of (1.6 mmol) of triethylamine and 0.2 ml (1.6 mmol) of phenyl chloroformate were used as starting materials. The crude product was crystallized from acetone, to obtain 210 mg of the final compound as white crystal. Yield: 33%. Melting point: 147.3° C. Rf=0.35 (CH₂Cl₂/MeOH, 95:5 v/v).

IR (KBr): 1740 (OC═O), 1716 (C═N), 1607 (C═Cₐᵣ) cm⁻¹

¹H NMR (200 MHz, CDCl₃, HMDS) δ ppm: 7.33 (s, 1H, NH), 7.12 and 6.74 (2d, 4H, J=8.62 and 8.60 Hz, aromatic H), 3.71 (s, 2H, CH₂—C═N), 3.14 and 2.56 (2s1, 8H, H of piperazine), 2.34 (t, 2H, J=7.34 Hz, CH₂—N), 1.47 (m, 2H, CH₂—C—N), 1.19 (s1, 30H, CH₂), 0.81 (t, 3H, J=6.05 Hz, CH₃).

### EXAMPLE 5

### Preparation of [4-(4′-octadecylpiperazine-1′-ylcarbonyl)benzylidene]-1,3-thiazolidine-2,4-dione

(Compound of formula (I) wherein D=Z=HET, HET=compound of formula (V), Z=CH═, Y=C═O, A=B=—CH₂—, and R=—(CH₂)₁₇—CH₃)

### 5-1: Preparation of 4-formylbenzoyl Chloride

In a 250 ml Erlenmeyer flask, 5 g (33 mmol) of 4-formylbenzoic acid was dissolved in 100 ml of chloroform. The mixture was stirred at 0° C., to which 3.63 ml (49 mmol) of thionyl chloride in 50 ml of chloroform was then added dropwise. After completion of the addition, the reaction mixture was heated at 40° C. for 3 hours. At the end of the reaction, the solvent was evaporated, and the resulting residue was taken up in dichloromethane, washed with Na₂CO₃ saturated solution, and then washed two times with water. The organic phase was dried rapidly over MgSO₄, filtered, and concentrated under vacuum. This yielded 4 g of the title acid chloride as colorless oil, which was used in a subsequent step without purification. Yield: 71%.

IR (KBr): 1779 (C═O, aldehyde), 1756 (ClC═O) cm⁻¹

¹H NMR (200 MHz, CDCl₃, HMDS) δ ppm: 10.10 (s, 1H, aldehyde H), 8.18 and 7.96 (2d, 4H, J=8.42 and 8.22 Hz, aromatic H).

### 5-2: Preparation of 1-(4′-formylbenzyl)-4-octadecylpiperazine

In a 250 ml Erlenmeyer flask equipped with an addition ampoule and a calcium chloride guard, 4 g (11 mmol) of octadecylpiperazine (prepared in the step 2-1 of Example 2) and 2.46 ml (17 mmol) of triethylamine were dissolved in 150 ml of anhydrous benzene. The mixture was stirred at 0° C., to which 2.99 g (17 mmol) of the acid chloride prepared in the above step 5-1 was then added dropwise. The mixture was stirred for 2 hours at ambient temperature. At the end of the reaction, the solvent was evaporated, and the residue was taken up in dichloromethane, washed with alkaline solution, and then washed two times with water. The organic phase was dried over MgSO₄, filtered, and concentrated under vacuum. The resulting crude product was purified by chromatography on a silica gel column using dichloromethane as eluent. This yielded 5.5 g of the title aldehyde as oil. Yield: 98%. Rf: 0.41 (CH₂Cl₂/MeOH, 97:3 v/v).

IR (KBr):1705 (C═O, aldehyde), 1642 (NC═O), 1609 (C═Cₐᵣ) cm⁻¹

¹H NMR (200 MHz, CDCl₃, HMDS) δ ppm: 10.02 (s, 1H, aldehyde H), 7.87 and 7.50 (2d, 4H, J=7.72 and 8.08 Hz, aromatic H), 3.86 and 3.48 (2s1, 4H, CH₂—N—C═O of piperazine), 2.67 (m, 4H, H of piperazine), 2.49 (t, 2H, J=7.66 Hz, CH₂—N), 1.51 (m, 2H, CH₂—C—N), 1.18 (s1, 30H, CH₂), 0.81 (t, 3H, J=6.16 Hz, CH₃).

### 5-3: Preparation of [5-(4′-octadecylpiperazin-1′-ylcarbonyl)benzylidene]-1,3-thiazolidine-2,4-dione

In a 100 ml round flask equipped with a cooler and a Dean & Starck apparatus, 4.2 g (8.9 mmol) of the aldehyde prepared in the above step 5-2, 1.04 g (8.8 mmol) of 2,4-thiazolidinedione and 0.5 g of pyridium benzoate were dissolved in 50 ml of toluene. The mixture was heated under reflux for 3 hours to remove moisture. At the end of the reaction, the toluene was evaporated, and the resulting residue was taken up in hot ethanol, followed by cooling the yellowish precipitate. The resulting crystal was filtered to produce 2.34 g of the final pure compound. Yield: 46%. Melting point: 86.3° C. Rf: 0.3(CH₂Cl₂/MeOH, 95:5 v/v).

IR (KBr): 1736 (NHC═O), 1700 (NC═O), 1604(C═Cₐᵣ) cm⁻¹

¹H NMR (200 MHz, CDCl₃, HMDS) δ ppm: 7.69 (s, 1H, CH═), 7.45 (s, 4H, aromatic H), 4.73 (s, 1H, NH), 3.79 and 3.46 (2s1, 4H, CH₂—N—C═O of piperazine), 2.48 (m, 4H, H of piperazine), 2.39 (t, 2H, J=7.33 Hz, CH₂—N), 1.45 (m, 2H, CH₂—C—N), 1.17 (s1, 30H, CH₂), 0.80 (t, 3H, J=5.89 Hz, CH₃).

### EXAMPLE 6

### Preparation of 1-[4′-(2,4-dioxo-1,3-thiazolidin-5-ylmethyl)benzoyl]-4-octadecylpiperazine

(Compound of formula (I) wherein D=Z-HET, HET=thiazolidinedione of formula (III), Z=—CH₂—, Y=C═O, A=B=—CH₂—, and R=—(CH₂)₁₇—CH₃).

A suspension of 210 mg (3.69×10⁻⁴ mol) of the compound prepared by Example 5 in 50 ml of absolute ethanol was hydrogenated in a Parr apparatus under pressure (40-50 psi) in the presence of 100% palladium black and hydrogen, and stirred for 5 hours at 60° C. At the end of the reaction, the palladium was filtered out, and the ethanol was evaporated. The resulting residue was purified by chromatography on a silica gel column using a dichloromethane/methanol mixture (99:1 v/v) as eluent. Then, the product was crystallized from acetonitrile to yield 126 mg of the final compound as light yellowish crystal. Yield: 60%. Melting point: 106.7° C. Rf: 0.55 (CH₂Cl₂/MeOH, 95:5 v/v).

IR (KBr): 1736 (NHC═O), 1700 (NC═O), 1604 (C═Cₐᵣ) cm⁻¹

¹H NMR (200 MHz, CDCl₃, HMDS) δ ppm: 7.5 (s1, 1H, NH), 7.30 and 7.20 (2d, 4H, J=8.15 and 8.18 Hz, aromatic H), 4.44 (s1, 1H, CH—C═O), 3.70 and 3.40 (2s1, 4H, CH₂—N—C═O of piperazine), 3.43 (dd, 2H, J=3.90 Hz, Ph-CH₂), 2.48 (m, 4H, H of piperazine), 2.39 (t, 2H, J=7.33 Hz, CH₂—N), 1.45 (m, 2H, CH₂—C—N), 1.17 (s1, 30H, CH₂), 0.80 (t, 3H, J=5.89 Hz, CH₃).

### EXAMPLE 7

### Preparation of 1-[4′-(4,5-dihydro-1,2,4(4H)-5-oxo-oxadiazol-3-ylmethyl)benzyl]-4-tetradecylpiperazin-2-one

(Compound of formula (I) wherein D=Z-HET, HET=oxadiazolone of formula (II), Z=—CH₂—, n=1, Y=A=—CH₂—, B=CO, and R=—(CH₂)₁₃—CH₃)

### 7-1: Synthesis of N-benzylaminoacetaldehyde Diethyl Acetal

A mixture consisting of 42 ml (0.2 mol) of aminoacetaldehyde diethyl acetal, 29.3 ml (0.2 mol) of benzaldehyde, 48 g of magnesium sulfate and 300 ml of toluene was heated under reflux for 6 hours. The solution was filtered and the solvent was evaporated. The residue obtained was used without purification. It was taken up in methanol, and 12 g (0.3 mol) of sodium borohydride was added slowly to the solution, and stirred for 30 minutes. After hydrolysis and the evaporation of the solvent, the resulting residue was dissolved in dichloromethane and washed with water. The organic phase was dried over MgSO₄, filtered, and evaporated under reduced pressure. The crude product was purified by flash chromatography using dichloromethane as eluent to produce 52 g of viscous oil. Yield: 81%. Rf: 0.34 (CH₂Cl₂/MeOH, 95:5 v/v).

IR (film): 3300 (NH), 1594 (C═Cₐᵣ) cm¹

¹H NMR (200 MHz, CDCl₃, HMDS) δ ppm: 7.23 (m, 5H, Hₐᵣ), 4.56 (t, 1H, J=5.58 Hz, CH), 3.75 (s, 2H, PhCH₂), 3.54 (m, 4H, OCH₂), 2.68 (d, 2H, J=5.58 Hz, CH₂), 1.71 (s, 1H, NH), 1.14 (t, 6H, J=7.04 Hz, CH₃).

### 7-2: Synthesis of (N-tetradecyl-N-benzyl)aminoacetaldehyde Diethyl Acetal

64 g (0.23 mol) of tetradecyl bromide was added to a mixture of 51.6 g (0.23 mol) of N-benzylaminoacetaldehyde diethyl acetal, 63.9 g (0.46 mol) of potassium carbonate and a catalytic amount of potassium iodide (1 g) in 700 ml of acetonitrile, and the mixture was heated under reflux overnight. The solution was filtered and the solvent was evaporated. The residue obtained was taken up in dichloromethane and washed with water. The organic phase was dried over MgSO₄, filtered, and evaporated under reduced pressure. The crude product was purified by chromatography using dichloromethane as eluent, to yield 88 g of the title compound as yellow oil. Yield: 90%. Rf: 0.65 (CH₂Cl₂/MeOH, 95:5 v/v).

IR (film): 1594 (C═Cₐᵣ) cm⁻¹

¹H NMR (200 MHz, CDCl₃, HMDS) δ ppm: 7.21 (m, 5H, Hₐᵣ), 4.47 (t, 1H, J=5.16 Hz, CH), 3.56 (s, 2H, PhCH₂), 3.50 (m, 4H, OCH₂), 2.55 (d, 2H, J=5.17 Hz, CH₂N), 2.41 (t, 2H, J=7.23 Hz, CH₂), 1.39 (t, 2H, J=6.87 Hz, CH₂), 1.13 (m, 28H, CH₂, CH₃), 0.81 (t, 3H, J=6.37 Hz, CH₃).

### 7-3: Preparation of N-tetradecylaminoacetaldehyde Diethyl Acetal

88 g (0.2 mole) of (N-tetradecyl-N-benzyl)aminoacetalaldehyde was dissolved in 300 ml of ethanol, followed by the addition of 20 mg of 10% Pd—C. The solution was subjected to hydrogenation under pressure at 40° C. for 48 hours. The catalyst was filtered out, and the solvent was evaporated under reduced pressure. The resulting residue was purified by chromatography using dichloromethane as eluent, to yield 59 g of yellow oil. Yield: 90%. Rf: 0.29 (CH₂Cl₂/MeOH, 95:5 v/v).

IR (film): 3300 (NH) cm⁻¹

¹H NMR (200 MHz, CDCl₃, HMDS) δ ppm: 4.59 (t, 1H, CH), 3.57 (m, 4H, CH₂O), 2.63 (d, 2H, J=5.57 Hz, CHCH₂), 2.58 (t, 2H, J=7.25 Hz, NHCH₂), 2.32 (1H, NH), 1.45 (t, 2H, J=7.03 Hz, CH₂), 1.15 (m, 28H, CH₃, CH₂), 0.81 (t, 3H, J=6.39 Hz, CH₃).

### 7-4: Preparation of Ethyl N-benzyloxycarbonylaminoacetate Ethyl

a) A mixture of 138 g (1 mol) of potassium carbonate and 70 g (0.5 mol) of glycine ethyl ester in 300 ml of tetrahydrofuran was stirred for 10 minutes and cooled to 0° C., followed by slow addition of 71 ml (0.5 mol) of benzyl chloroformate. The solution was stirred for 30 minutes and filtered, followed by evaporation of the solvent. The resulting residue was dissolved in dichloromethane and washed with water. The organic phase was dried over MgSO₄ and filtered, and the solvent was evaporated under reduced pressure. The residue obtained was used in a subsequent step without purification.

IR (film): 1735 (—O—CO—), 1750 (—O—CO—N) cm⁻¹

¹H NMR (200 MHz, CDCl₃, HMDS) δ ppm: 7.21 (m, 5H, Hₐᵣ), 5.81 (s1, 1H, NH), 5.01 (s, 2H, PhCH₂), 4.06 (q, 2H, J=7.14 Hz, CH₂CH₃), 3.80 (d, 2H, J=14.27 Hz, NHCH₂), 1.14 (t, 3H, J=7.13 Hz, CH₃).

b) The residue obtained in the above step, which had been dissolved in 250 ml of ethanol, was treated with 10% potassium carbonate solution and heated under reflux overnight. The ethanol was evaporated, and the aqueous phase was acidified (pH=1) with concentrated HCl. The precipitate obtained was filtered and dried to produce 105 g of a white solid. Yield: 90%. Melting point: 110° C. Rf: 0.25 (CH₂Cl₂/MeOH, 90:10 v/v).

IR (KBr): 1678 (C═C), 1727(OC═O) cm⁻¹

¹H NMR (200 MHz, CDCl₃, HMDS) δ ppm: 7.24 (m, 5H, Hₐᵣ), 5.23 (s1, 1H, NH), 5.06 (s, 2H, CH₂, PhCH₂), 4.63 (s, 1H, OH), 3.94 (d, 2H, J=5.50 Hz, CH₂COOH).

### 7-5: Preparation of N-benzyloxycarbonylamino-N′-2,2-diethoxyethyl-N′-tetradecylacetamide

To a mixture of 18.7 g (5.6 mmol) of tetradecylaminoacetaldehyde diethyl acetal, 12.6 g (56 mmol) of ethyl N-benzyloxycarbonylacetate, 15 ml (0.112 mol) of triethylamine and 9 g (67 mmol) of 1-hydroxybenzotriazole in 120 ml of dichloromethane, 24.7 g (0.12 mol) of N,N′-dicyclohexylcarbodiimide was added. After heating to reflux for 2 hours, the solution was filtered and washed with water. The organic phase was dried over MgSO₄, filtered and evaporated. The resulting residue was purified on a silica gel column using a mixture of CH₂Cl₂/MeOH (99:1 v/v), to yield 25 g of colorless oil. Yield: 96%. Rf: 0.42 (CH₂Cl₂/MeOH, 98:2 v/v).

IR (film): 1649 (C═O), 1720 (OC═O) cm⁻¹

¹H NMR (200 MHz, CDCl₃, HMDS) δ ppm: 7.17 (m, 5H, Hₐᵣ), 5.85 (s1, 1H, NH), 5.01 (s, 2H, PhCH₂), 4.54 (t, 1H, J=5.26 Hz, CH), 3.92-4.02 (m, 2H, NHCH₂CO), 3.48-3.65 (m, 2H, NCH₂CH), 3.2 (m, 6H, CH₂, OCH₂), 1.44 (s1, 2H, CH₂), 1.17 (s1, 22H, CH₂), 1.09 (t, 6H, J=6.98 Hz, CH₃), 0.79 (t, 3H, J=6.2 Hz, CH₃).

### 7-6: Preparation of 4-benzyloxycarbonyl-1-tetradecylpiperazin-2-one

23 g (47 mmol) of the amide prepared in the above step, which had been dissolved in 250 ml of toluene, was added with a catalytic amount (780 mg, 4.1 mmol) of paratoluene sulfonic acid at ambient temperature. The solution was stirred at 75° C. for 3 hours. It was cooled and washed with water, and the organic phase was dried over MgSO₄, filtered and concentrated. The resulting residue was purified by chromatography using dichloromethane as eluent, to yield 14 g of yellow oil. Yield: 72%. Rf: 0.61 (CH₂Cl₂/MeOH, 98:2 v/v).

IR (film): 1669 (C═O, C═C), 1700 (OC═O) cm⁻¹

¹H NMR (200 MHz, CDCl₃, HMDS) δ ppm: 7.25 (m, 5H, Hₐᵣ), 6.29 (dd, 1H, J=21.58 and 5.98 Hz, CH═CH), 5.42 (dd, 1H, J=18.78 and 6.01 Hz, CH═CH), 5.12 (s, 2H, PhCH₂O), 4.59 (s, 2H, COCH₂N), 3.39 (t, 2H, J=7.24 Hz, CH₂), 1.33 (s1, 2H, CH₂), 1.18 (s1, 22H, CH₂), 0.81 (t, 3H, J=6.32 Hz, CH₃)

### 7-7: Preparation of 1-tetradecylpiperazin-2-one Chlorohydrate

To a solution of 10.5 g (24 mmol) of 4-benzyloxycarbonyl-1-tetradecylpiperazin-2-one dissolved in 100 ml of ethanol, 5 ml of concentrated HCl was added. The mixture was subjected to catalytic hydrogenation under hydrogen pressure in the presence of 1 g of 10% Pd—C at 40° C. for 24 hours. After filtration, the solvent was evaporated, and the residue was crystallized from ether to yield 7 g of a yellow solid. Yield: 86%. Melting point: 161.6° C. (decomposition). Rf: 0.25 (CH₂Cl₂/MeOH, 90:10 v/v).

IR (KBr): 1655(C═O), 3451 (NH₂) cm⁻¹

¹H NMR (200 MHz, CDCl₃, HMDS) δ ppm: 9.87(s1, 1H, NH₂), 7.7-8.9 (s1, 1H, NH₂), 4.18 (s1, 2H, NCH₂), 3.93 (s1, 2H, NCH₂), 3.63 (s1, 2H, NCH₂), 3.30 (s, 2H, CH₂), 1.46 (s1, 2H, CH₂), 1.19 (s1, 22H, CH₂), 0.81 (s, 3H, CH₃).

### 7-8: Preparation of 4-(4-cyanomethylbenzyl)-1-tetradecylpiperazin-2-one

2.2 g (10 mmol) of 4-bromomethylphenylacetonitrile was added to a mixture of 2.95 g (8.8 mmol) of 1-tetradecylpiperazin-2-one chlorohydrate, 2.6 g (17.6 mmol) of potassium carbonate and 0.5 g of potassium iodide in 100 ml of acetonitrile. The solution was stirred under reflux for 4 hours, filtered and evaporated. The resulting residue was dissolved in dichloromethane and washed with sodium carbonate saturated solution. The organic phase was dried over MgSO₄, filtered and evaporated. The crude product obtained was purified by flash chromatography with a mixture of CH₂Cl₂/MeOH (99:1 v/v), to yield 3.6 g of yellow oil. Yield: 95%. Rf: 0.48 (CH₂Cl₂/MeOH, 95:5 v/v).

IR (film): 1647 (C═O), 2249 (CN) cm⁻¹

¹H NMR (200 MHz, CDCl₃, HMDS) δ ppm: 7.19 (m, 4H, Hₐᵣ), 3.67 (s, 2H, PhCH₂CN), 3.47 (s, 2H, NCH₂Ph), 3.26 (m, 4H, NCH₂), 3.07 (s, 2H, COCH₂N), 2.58 (t, 2H, J=5.39 Hz, CH₂), 1.47 (s1, 2H, CH₂), 1.18 (s1, 22H, CH₂), 0.81 (t, 3H, J=6.40 Hz, CH₃)

### 7-9: Preparation of 4-[4-(N-hydroxyamidinomethyl)benzyl]-1-tetradecylpiperazin-2-one

To a mixture of 7 g (50 mmol) of potassium carbonate and 2.9 g (41 mmol) of hydroxylamine chlorohydrate in 80 ml of ethanol, which had been heated under reflux, 3.6 g (8.4 mmol) of 4-(4-cyanomethylbenzyl)-1-tetradecylpiperazin-2-one was added dropwise. After completion of the addition, the mixture was heated under reflux for 12 hours. After filtration and the evaporation of solvent, the resulting residue was taken up in dichloromethane and washed with water. The organic phase was dried over MgSO₄, filtered and evaporated. The residue was purified on a silica gel column with a mixture of CH₂Cl₂/MeOH (98:2 v/v), to yield 2.2 g of yellow oil. Yield: 56%. Rf: 0.43 (CH₂Cl₂/MeOH, 90:10 v/v).

IR (film): 1636 (C═O, C═N) cm⁻¹

¹H NMR (200 MHz, CDCl₃, HMDS) δ ppm: 7.17 (m, 4H, Hₐᵣ), 4.51 (s, 2H, NH₂), 3.44 (s, 2H, PhCH₂CN), 3.35 (s, 2H, NCH₂Ph), 3.24 (m, 4H, NCH₂), 3.05 (s, 2H, COCH₂N), 2.58 (t, 2H, J=5.21 Hz, CH₂), 1.45 (s1, 2H, CH₂), 1.18 (s1, 22H, CH₂), 0.80 (t, 3H, J=6.38 Hz, CH₃).

### 7-10: Preparation of 1-[4′-(4,5-dihydro-1,2,4(4H)-5-oxo-oxadiazol-3-ylmethyl)benzyl]-4-tetradecylpiperazin-2-one

To a solution of 1.1 g (2.4 mmol) of 4-[4-(N-hydroxyamidinomethyl)bebzyl]-1-tetradecylpiperazin-2-one and 0.4 ml (2.9 mmol) of triethylamine in 130 ml of dichloromethane, which had been cooled at 0° C. for 15 minutes, 0.36 ml (2.8 mmol) of phenyl chlorocarbonate was added dropwise. The mixture was stirred for 2 hours, treated with a saturated solution of Na₂CO₃ and washed with water. The organic phase was dried over MgSO₄, filtered and concentrated. The residue obtained was dissolved in 50 ml of toluene and heated under reflux for 6 hours. The toluene was evaporated and the resulting residue was purified by chromatography on a silica gel column with CH₂Cl₂/MeOH (98:1 v/v) as eluent, and then crystallized from ether, to yield 300 mg of a yellow solid. Yield: 30%. Rf: 0.52 (CH₂Cl₂/MeOH, 90:10 v/v).

IR (KBr): 1636 (CON), 1775 (—O—CO—N) cm⁻¹

¹H NMR (200 MHz, CDCl₃, HMDS) δ ppm: 7.16 (m, 4H, Hₐᵣ), 5.23 (s, 1H, NH), 3.76 (s, 2H, PhCH₂CN), 3.38 (s, 2H, NCH₂Ph), 3.25 (m, 4H, CH₂N), 2.86 (s, 2H, COCH₂N), 2.57 (t, 2H, J=5.23 Hz, CH₂), 1.45 (m, 2H, CH₂), 1.18 (s1, 22H, CH₂), 0.81 (t, 3H, J=6.42 Hz, CH₃).

### EXAMPLE 8

### Preparation of 1-[4′-(4,5-dihydro-1,2,4(4H)-5-oxo-oxadiazol-3-ylmethyl)benzoyl]-4-tetradecylpiperazine

(Compound of formula (I) wherein D=Z-HET, HET=oxadiazolone of formula (II), Z=—(CH₂)₂—, n=2, A=B=—CH₂—, Y=CO, R=—(CH₂)₁₃—CH₃)

### 8-1: Preparation of 4-(2-chloro-2-cyanoethyl)benzoic Acid

In a 250 ml Erlenmeyer flask, 10 g (72 mmol) of para-aminobenzoic acid was dissolved in 70 ml of acetic acid, to which 6 ml of 12 N hydrochloric acid was then added. The mixture was cooled to 0° C., and 2.5 g (36.2 mmol) of NaNO₂ was added in portions. After stirring for 30 minutes, the viscous liquid obtained was added dropwise to a mixture of 6.5 ml (94.8 mmol) of acrylonitrile and several milligrams of copper oxide (CuO), which had been suspended in 20 ml of acetone anhydride. The reaction mixture was stirred at ambient temperature for two hours, and the solid obtained was filtered under vacuum and washed several times with water. The crude product was purified by recrystallization from water, to yield a white solid. Yield: 65%. Melting point: 157° C. Rf: 0.29 (CH₂Cl₂/MeOH, 50:50 v/v).

IR (KBr): 1690 (C═O), 2240 (CN) cm⁻¹

¹H NMR (200 MHz, CDCl₃, HMDS) δ ppm: 7.9 (d, 2H, J=8.22 Hz, Hₐᵣ), 7.4 (d, 2H, J=8.18 Hz, Hₐᵣ), 5.5 (t, 1H, J=7 Hz, CNCHCl), 3.43 (d, 2H, J=6.96 Hz, CH₂CHCN).

### 8-2: Preparation of 4-(2-cyanoethyl)benzoic Acid

To 10 g (47 mmol) of 4-(2-chloro-2-cyanoethyl)benzoic acid dissolved in 250 ml of glacial acetic acid, 1.56 g (23 mmol) of zinc powders were added in portions. The mixture was heated under reflux for two hours. The salt (ZnCl₂) formed was filtered under vacuum and washed several times with water. A precipitate which had been formed at low temperature in the filtrate was filtered, washed several times with water, and dried. This yielded a white solid. Yield: 68%. Melting point: 165° C. Rf: 0.25 (CH₂Cl₂/MeOH, 50:50 v/v).

IR (KBr): 1700 (C═O), 2252 (CN) cm⁻¹

¹H NMR (200 MHz, CDCl₃, HMDS) δ ppm: 7.9 (d, 2H, J=8.17 Hz, Hₐᵣ), 7.25 (d, 2H, J=8.13 Hz, Hₐᵣ), 2.95 (t, 2H, J=7.22 Hz, CH₂CN), 2.6 (t, 2H, J=7.36 Hz, CH₂CH₂CN).

By the condensation steps, which had been performed in the same manner as the experimental protocol of Example 2 above, substantially the same yield was obtained and a final product with oxadiazolone as named above was collected.

### EXAMPLE 9

### Preparation of 1-[4′-(4,5-dihydro-1,2,4(4H)-5-oxo-oxadiazol-3-ylpropyl)benzoyl]-4-tetradecylpiperazine

(Compound of formula wherein D=Z-HET, HET=oxadiazolone of formula (II), Z=—(CH₂)₃—, n=3, A=B=—CH₂—, Y=CO, and R=—(CH₂)₁₃—CH₃)

### 9-1: Preparation of 1-bromo-3-phenylpropane

To a solution of 10 g (73 mmol) of 3-phenylpropan-1-ol in 150 ml of anhydrous dichloromethane, 50 ml of a solution of 1M PBr₃ (36 mmol) was added in portions. The reaction mixture was stirred at ambient temperature for one hour. After washing several times with water, the organic phase was dried and evaporated. The residue obtained was purified by chromatography on silica gel with a mixture of ether/petroleum ether (5:95 v/v) as eluent, to give the bromide derivative as viscous liquid. Yield: 80%. Rf: 0.25 (ether/petroleum ether, 5:95 v/v).

IR (film): 1605 (C═Cₐᵣ) cm⁻¹

¹H NMR (200 MHz, CDCl₃ HMDS) δ ppm: 7.25-7.06 (m, 5H, Hₐᵣ), 3.29 (t, 2H, J=6.59 Hz, CH₂Br), 2.68 (t, 2H, J=7.34 Hz, PhCH₂CH₂CH₂Br), 2.14-1.99 (m, 2H, CH₂CH₂CH₂Br).

### 9-2: Preparation of 4-(3-bromopropyl)acetophenone

To a mixture solution of 17.5 g (88 mmol) of aluminum trichloride and 50 ml of acetyl chloride in 100 ml of CS₂, 25 g (125 mmol) of a solution of the bromide derivative 1-bromo-3-phenylpropane solution in 20 ml of acetyl chloride was added dropwise at 0° C. The mixture was stirred at ambient temperature for two hours. An excess of acetyl chloride and CS₂ were removed by evaporation under reduced pressure. The residue obtained was taken up in dichloromethane, washed several times with water, dried over MgSO₄, and then concentrated under vacuum. Evaporation under reduced pressure yielded the title substituted acetophenone as yellow liquid. Yield: 79%. Boiling point: 140-145° C./3 mmHg. Rf: 0.25 (ether/petroleum ether, 50:50 v/v).

IR (film): 1670 (C═O), 1605 (C═Cₐᵣ) cm⁻¹

¹H NMR (200 MHz, CDCl₃, HMDS) δ ppm: 7.79 (s1, 2H, Hₐᵣ), 7.23 (s1, 2H, Hₐᵣ), 3.26 (t, 2H, J=6.49 Hz, CH₂Br), 2.7 (t, 2H, J=6.26 Hz, PhCH₂CH₂CH₂Br), 2.48 (s, 3H, CH₃), 2.08-1.9 (m, 2H, CH₂CH₂CH₂Br).

### 9-3: Preparation of 4-(3-bromopropyl)benzoic Acid

To 33 g of NaOH solution in 200 ml of water, 50 ml of Br₂ and 100 ml of dioxane were successively added dropwise. The mixture was cooled to 0° C., and 22 g of 4-(3-bromopropyl)acetophenpone was added dropwise. Stirring was maintained at ambient temperature until the brown color of bromide (one hour) disappeared. The mixture was carefully acidified with an aqueous solution of 12 N (20 ml) of HCl. The precipitate formed was filtered under vacuum, rinsed several times with water and dried, to produce a yellow solid. Yield: 85%. Melting point: 120° C. Rf: 0.25 (MeOH/CH₂Cl₂, 20:80 v/v).

IR (KBr): 3340 (OH), 1700 (C═O) cm⁻¹

¹H NMR (200 MHz, CDCl₃, HMDS) δ ppm: 7.9 (d, 2H, J=8.16 Hz, Hₐᵣ), 7.2 (d, 2H, J=8.3 Hₐᵣ), 3.3 (t, 2H, J=6.47 Hz, CH₂Br), 2.78 (t, 2H, J=7.7 Hz, Ph CH₂CH₂CH₂Br), 2.18-2.04 (m, 2H, CH₂CH₂CH₂Br).

### 9-4: Preparation of 4-(3-cyanopropyl)benzoic Acid

The bromide derivative prepared in the above step was converted into nitrile by the same protocol as described in Example 1 above. Yield: 75%. Viscous appearance. Rf: 0.29 (MeOH/CH₂Cl₂, 15:85 v/v).

IR (film): 3345 (OH), 1700 (C═O), 2253 (CN) cm⁻¹

¹H NMR (200 MHz, CDCl₃, HMDS) δ ppm: 7.9 (d, 2H, J=8.09 Hz, Hₐᵣ), 7.2 (d, 2H, J=8.09 Hz, Hₐᵣ), 2.8 (t, 2H, J=7.44 Hz, CH₂CN), 2.28 (t, 2H, J=6.99 Hz, PhCH₂CH₂CH₂CN), 2.02-1.91 (m, 2H, CH₂CH₂CH₂CN).

### 9-5: Subsequent Step

The concentration of the tetradecylpiperazine on 3-chloromethylbenzoyl chloride was performed in basic medium in the same manner as in Example 2 above, to yield a corresponding chloride. According to the same protocol as described above, it was converted into nitrile, and then into amidoxime and finally into oxadiazolone: 1-[4′-(4,5-dihydro-1,2,4(4H)-5-oxo-oxadiazol-3-ylpropyl)benzoyl]-4-tetradecylpiperazin-2-one. The characteristics of all the intermediates and final products thus obtained are given below.

### 1-(3-chloromethylbenzoyl)-4-tetradecylpiperazine

Appearance: viscous oil. Yield: 67%. Eluent (MeOH/CH₂Cl₂, 5:95 v/v). Rf: 0.25 (MeOH/CH₂Cl₂, 10:90 v/v).

IR (film): 1660 (NCO), 1610 (C═Cₐᵣ) cm⁻¹

¹H NMR (200 MHz, CDCl₃, HMDS) δ ppm: 7.38 (s1, 4H, Hₐᵣ), 4.55 (s, 2H, CH₂Cl), 3.72 (m, 2H, NCH₂), 3.38 (m, 2H, NCH₂), 2.52 (m, 4H, NCH₂), 2.36 (t, 2H, NCH₂), 1.41 (m, 2H, NCH₂CH₂), 1.25-1.15 (s1, 22H, CH₂), 0.81 (t, 3H, J=6.09 Hz, CH₃).

### 1-(3-cyanomethylbenzoyl)-4-tetradecylpiperazine

Appearance: viscous oil. Yield: 67%. Eluent (MeOH/CH₂Cl₂, 5:95 v/v). Rf: 0.25 (MeOH/CH₂Cl₂, 10:90 v/v).

IR (film): 1665 (NCO), 2253 (CN), 1605 (C═Cₐᵣ) cm⁻¹

¹H NMR (200 MHz, CDCl₃, HMDS) δ ppm: 7.36-7.23 (s1, 4H, Hₐᵣ), 3.71 (s, 2H, CH₂CN), 3.74-3.71 (m, 2H, NCH₂), 3.43 (m, 2H, NH₂), 2.54-2.25 (m, 4H, NCH₂), 2.29 (t, 2H, J=7.45 Hz, NCH₂), 1.5-1.35 (m, 2H, NCH₂CH₂), 1.3-1.1 (m, 22H, CH₂), 0.81 (t, 3H, J=6.36 Hz, CH₃).

### 1-[3-(N-hydroxyamindino)methylbenzoyl]-4-tetradecylpiperazine

Appearance: viscous oil. Yield: 58%. Eluent (MeOH/CH₂Cl₂, 5:95 v/v). Rf: 0.25 (MeOH/CH₂Cl₂, 10:90 v/v).

IR (film): 3430 (NH), 1660 (NCO), 1605 (C═Cₐᵣ) cm⁻¹

¹H NMR (200 MHz, CDCl₃, HMDS) δ ppm: 7.24-7.07 (m, 4H, Hₐᵣ), 3.65-3.53 (m, 2H, NCH₂), 3.33-3.28 (m, 2H, NCH₂), 2.41-2.11 (m, 6H, NCH₂), 2.09 (s, 2H, CH₂CN), 1.5-1.3 (m, 2H, NCH₂CH₂), 1.25-1.15 (m, 22H, CH₂), 0.81 (t, 3H, J=6.41 Hz, CH₃).

### 1-[3-(4,5-dihydro-1,2,4(4B)-oxadiazol-5-one-3-yl)methylbenzoyl]-4-tetradecylpiperazine

Appearance: Viscous oil. Yield: 67%. Eluent (MeOH/CH₂Cl₂, 5:95 v/v). Rf: 0.25 (MeOH/CH₂Cl₂, 10:90 v/v).

IR (film): 3435 (NH), 1665 (NCO), 1610 (C═Cₐᵣ) cm⁻¹

¹H NMR (200 MHz, CDCl₃, HMDS) δ ppm: 7.3-7.2 (m, 4H, Hₐᵣ), 6.57 (s1, 1H, NH), 3.68-3.35 (m, 4H, NCH₂), 2.52-2.29 (m, 6H, NCH₂), 2.1 (s, 2H, CH₂CN), 1.4-1.35 (m, 2H, NCH₂CH₂), 1.3-1.1 (m, 22H, CH₂), 0.8 (t, 3H, J=6 Hz, CH₃).

### EXAMPLE 10

### Preparation of 1-[4′-(4,5-dihydro-1,2,4(4H)-5-oxo-oxadiazol-3-yl-methylbenzoyl)-4-(N-octadecylaminocarbonyl)piperazine

(Compound of formula (I) wherein D=Z-HET, HEt=oxadiazolone of formula (II), Z=—(CH₂)—, n=1, A=B=—CH₂—, Y=CO, and R=—(CH₂)₁₇—CH₃)

### 10-1: Preparation of N-octadecylaminocarbonylpiperazine

In a 250 ml Erlenmeyer flask, 13 g (0.151 mol) of piperazine dissolved in 100 ml of dichloromethane was stirred. 4.42 g (15 mmol) of 1-octadecylisocyanate was added to the solution, and stirring was maintained at ambient temperature for one hour. At the end of the reaction, the solution was washed two times with water. The organic phase was dried over MgSO₄, filtered, and evaporated, to yield 5.21 g of white crystal.

Yield: 91%. Melting point: 72° C. Rf: 0.46 (CH₂Cl₂/MeOH/NH₄OH, 80:20:2 v/v/v).

IR (KBr): 3364 (NH), 1620 (N—CO—N) cm⁻¹

¹H NMR (200 MHz, CDCl₃, HMDS) δ ppm: 4.68 (t, 1H, J=5.01 Hz, NHCO), 3.26 (t, 4H, J=5.22 Hz, CH₂NCO), 3.14 (q, 2H, J=7.14 Hz, CH₂NHCO), 2.77 (t, 4H, J=5.21 Hz, CH₂NH), 1.73 (s1, 1H, NH), 1.42 (m, 2H, CH₂CH₂NH), 1.19 (s1, 30H, CH₂), 0.81 (t, 3H, J=6.20 Hz, CH₃).

### 10-2: Preparation of 1-(4′-chloromethylbenzoyl)-4-(N-octadecylaminocarbonyl)piperazine

This intermediate was obtained under the same conditions as in Example 2 above using 9.9 g (26 mmol) of N-octadecylcarbonylpiperazine, 5.4 ml (38 mmol) of triethylamine and 5 g (26 mmol) of 4-chloromethylbenzoic acid chloride as starting materials. The purification by chromatography on silica gel with dichloromethane as eluent yielded 12.2 g of the title product as white crystal. Yield: 88%. Melting point: 68-70° C. Rf: 0.4 (CH₂Cl₂/MeOH, 95:5 v/v).

IR (KBr): 2365 (CN), 1653 (CON), 1730 (C═Cₐᵣ) cm⁻¹

¹H NMR (200 MHz, CDCl₃, HMDS) δ ppm: 7.30-7.39 (s1, 4H, Hₐᵣ), 4.82 (t, 1H, J=5 Hz, NHCON), 4.48 (s, 2H, PhCH₂Cl), 3.52 (s1, 4H, CH₂NCONH), 3.34 (s1, 4H, CH₂NCO), 3.15 (q, 2H, J=7 Hz, CH₂NH), 1.43 (m, 2H, CH₂CH₂NH), 1.19 (sm, 30H, CH₂), 0.81 (t, 3H, J=5.15 Hz, CH₃).

### 10-3: Preparation of 1-(4′-cyanomethylbenzoyl)-4-(N-octadecylaminocarbonyl)piperazine

This compound was obtained by the same synthetic protocol as described in Example 2 above using 5.33 g (10 mmol) of 1-(4′-chloromethylbenzoyl)-4-(octadecylaminocarbonyl)piperazine and 1.96 g (40 mmol) of sodium cyanide as starting materials. Thus, 3.84 g of a white precipitate was obtained. Yield: 74%. Melting point: 90° C. Rf: 0.56 (CH₂Cl₂/MeOH, 93:7 v/v).

IR (KBr): 2365 (CN), 1653 (CON), 1730 (C═Cₐᵣ) cm⁻¹

¹H NMR (200 MHz, CDCl₃, HMDS) δ ppm: 7.30-7.39 (s1, 4H, Hₐᵣ), 4.53 (t, 1H, J=5 Hz, NHCON), 3.73 (s, 2H, PhCH₂CN), 3.52 (s1, 4H, CH₂NCONH), 3.34 (s1, 4H, CH₂NCO), 3.15 (q, 2H, J=7 Hz, CH₂NH), 1.43 (m, 2H, CH₂CH₂NH), 1.19 (s1, 30H, CH₂), 0.81 (t, 3H, J=5.15 Hz, CH₃).

### 10-4: Preparation of 1-[4′-(N-hydroxyamidinomethyl)benzoyl]-4-(N-octadecylaminocarbonyl)piperazine

This amidoxime was obtained under the same conditions as described above using 5.8 g (84 mmol) of hydroxylamine chlorohydrate, 14.07 g (102 mmol) of potassium carbonate and 8.9 g (17 mmol) of 1-(4′-cyanomethylbenzoyl)-4-(octadecylaminocarbonyl)piperazine as starting materials. The residue obtained was purified by chromatography on silica gel with a mixture of CH₂Cl₂/MeOH (98:2 v/v) as eluent. Thus, 2.36 g of white crystal was obtained. Yield: 38%. Melting point: 104-106°° C. Rf: 0.43 (CH₂Cl₂/MeOH, 90:10 v/v).

IR (KBr): 3493 (OH), 3355 (NH₂), 2200 (CN), 1615 (C═Cₐᵣ) cm⁻¹

¹H NMR (200 MHz, CD₃OD, HMDS) δ ppm: 7.35 and 7.30 (2d, 4H, J=8.8 and 8.5 Hz, Hₐᵣ), 4.7 (t, 1H, J=6 Hz, NHCON), 4.8 (s, 2H, NH₂), 3.61 (m, 4H, CH₂NCONH), 3.50 (s, 2H, PhCH₂), 3.27 (m, 4H, CH₂NCO), 3.07 (t, 2H, J=6 Hz, CH₂NH), 1.39 (m, 2H, CH₂CH₂NH), 1.21 (s1, 30H, CH₂), 0.83 (t, 3H, J=8 Hz, CH₃).

### 10-5: Preparation of 1-[4′-(4,5-dihydro-1,2,4(4H)-5-oxo-oxadiazol-3-yl-methylbenzoyl)-4-(N-octadecylaminocarbonyl)piperazine

This synthesis was performed in two steps by the same protocol as described in Example 2 using 1.25 g (2.3 mmol) of the amidoxime as described above, 0.38 ml (2.75 mmol) of triethylamine and 0.34 ml (2.75 mmol) of phenyl chlorocarbonate as starting materials. 0.8 g of white crystal was obtained after chromatography on silica gel with dichloromethane as eluent. Yield: 59%. Melting point: 150-152° C. Rf: 0.43 (CH₂Cl₂/MeOH, 93:7 v/v).

IR (KBr): 1780 (OCON), 1618 (C═N), 1550 (C═Cₐᵣ) cm⁻¹

¹H NMR (200 MHz, CD₃OD, HMDS) δ ppm: 7.36 (s, 4H, Hₐᵣ), 4.94 (t, 1H, J=5.53 Hz, NHCON), 3.77 (s, 2H, PhCH₂), 3.56 (s1, 2H, CH₂NCONH), 3.27 (s1, 2H, CH₂NCO), 3.07 (q, 2H, J=7.37 Hz, CH₂NHCO), 1.41 (s1, 2H, CH₂CH₂NHCO), 1.18 (s1, 30H, CH₂), 0.81 (t, 3H, J=6.8 Hz, CH₃).

### EXAMPLE 11

### In Vitro Biological Activity Assay

Phospholipase A2 hydrolyzes an ester linkage at the sn-2 position of glycerophospholipid and liberates fatty acids and lysophospholipids. The in vitro action of particular compounds was evaluated by the analysis of fatty acids according to the fluorometric method described in Radvanyi et al., Anal. Biochem. 1989, 177. 103-109, and by the analysis of lysophospholipids according to the UV spectrophotometric method described in Reynolds et al., Anal. Biochem. 1992, 204, 190-197.

### 11-1: Material and Method

### 11-1-1: Materials

Enzymes used were two secretory enzymes of group II (human recombinant PLA₂, and PLA₂ basic subunit isolated from Crotalus durissus terrificus), and a secretory enzyme of porcine pancreatic (group I) PLA₂.

Regarding substrates, palmitoyl-2-(10-pyrenyl decanoyl)-sn-glycero-3-phosphatidylglycerolic acid as a fluorescent substrate was used in the fluorometric method, and the lithium salt of 1,2-bis-(dihexanylthio)-dideoxy-rac-glycero-3-phosphorylglycerol was used in the UV spectrophotometric method.

The fluorometric analysis was performed using a Perkin Elmer LS50 luminescence spectrometer in a unit dosage polystyrene cell having a size of 1 cm. The exact concentration of the fluorescent substrate was determined by UV Unicam spectrometry in a quartz cell.

The UV spectrophotometric analysis was performed on an ELx 808 Ultra Micro Plate Reader apparatus (96-well plate).

### 11-1-2: Methods

a) Fluorometric Analysis

PLA₂ is an enzyme that hydrolyzes an ester linkage at the sn-2 position of phospholipid. In an aggregated form, the fluorescent substrate shows the maximum fluorescence emission at 490 nm, but does not show fluorescence emission at 398 nm. After hydrolysis with the enzyme, fluorescence emitted by the liberated fatty acid (pyrenyl decanoic acid) complex with bovine serum albumin (BSA) is increased, and strong fluorescence emission is observed at 378 and 398 nm. The principle of the analysis is to measure a difference in fluorescence at 398 nm in order to examine the production of fatty acids liberated for a given period of time, thus determining the PLA₂ activity.

Measurement of the enzymatic activity was performed in a cell containing: 960 μl of Tris buffer; 50 mM HCl (pH 7.5); 0.5 M NaCl, 1 mM EGTA; and 1 μl substrate. This mixture was stirred under reflux for one minute to permit the formation of vesicles of substrate, and then, 10 μl of 10% SAB, 10 μl of solvent (ethanol or DMSO) or inhibitor solution, 10 μl of PLA₂ to a given concentration and finally 10 μl of 1 M calcium chloride (CaCl₂) for initiating the activity were successively added to the mixture under stirring.

Good conditions of measurement of the enzymatic activity include saturation of the enzyme, and thus, initial concentrations used are as follows: (i) human recombinant PLA₂: 0.1 μl/ml; (ii) porcine pancreatic PLA₂: 0.6 μl/ml; and (iii) Crotalus durissus terrificus (CB) PLA₂: 0.05 μl/ml. Mother solution containing the inhibitor was prepared at an initial concentration of 10⁻² M.

The enzymatic activity is shown by a curve of which the original slope permits to calculate the initial velocity of the reaction. The following equation permits to calculate the enzymatic activity (A; μmol) of fatty acids liberated per minute. In the equation, S₀ represents the slope of a curve in the absence of calcium (control), S the slope in the presence of calcium, V the volume (μl) of the substrate solution, and Fₘₐₓ the signal of maximum fluorescence obtained at the end of the enzymatic reaction:

$$A = {2.10}^{- 4} \times \frac{( S - {S}_{0} ) \times V}{{F}_{max}}$$

The residual activity in the presence of an inhibitor was evaluated by slopes obtained in the absence and presence of an inhibitor, according to the following equation:
Residual activity (%)=(S−S₀) in the presence of inhibitor/(S−S₀) in the absence of inhibitor

Values obtained as a logarithmic function of the inhibitor concentrations used permit to determine the IC₅₀ value, i.e., the inhibitor concentration required to cause 50% reduction of the enzymatic activity. The lower the IC₅₀ value, the higher the inhibitory activity of a compound tested.

PLA₂ has a higher affinity for organized substrates. However, the following three reasons will be explained for the inhibition observed eventually:

(1) The inhibitor destroys micelles of substrate and renders substrate inaccessible to the enzyme. In this case, the inhibition is due to the unavailability of substrate.

(2) A portion of the inhibitor can fix vesicles of substrate, such that the IC₅₀ value is estimated.

(3) The inhibitor agent will react with an active site group or with another portion of the enzyme to interfere with the hydrolysis of substrate. In this case, the inhibition observed is remarkable, occurs at the level of an active site, and can present or not present a reversible characteristic.

The fluorometric test is a very sensible technique, but permits to distinguish the difference between three types of inhibition, and substrate will be in a micellar form. On the other hand, in a spectrophotometric test, which will be described below, the monomeric state of substrate permits to level ambiguity for the reality of the inhibition, although in this test, the enzyme will not completely function under such optimal conditions.

b) UV Spectrophotometric Analysis

Lysothiophospholipid (LTPL) liberated by the lypolytic action of PLA₂ in the presence of calcium reacts with dithionitrobenzoic acid (DTNB) present in medium to form an LTPL-TNB complex and an TNB-anion which induces the yellowing of reaction medium. The measurement of optical density at 412 nm (absorbance wavelength of TNB-ion) shows the production of lysothiophospholipid and the PLA₂ activity.

The measurement of enzymatic activity is performed in a multiple well plate of which each well contains 190 μl of 1× buffer, 2 μl of 10 mM DTNB, 2 θl of 20 mM substrate, 2 μl of solvent or inhibitor solution, and 2 μl of PLA₂ at a given concentration. The plate was stirred and 2 μl of 1 M calcium chloride was added to initiate the enzymatic reaction. Substrate was used at a lower concentration than micellar critical concentration (about 1 mM) in a monomeric form, and the ratio of substrate to enzyme was respected. This justifies the utilization of substrate at five times lower concentration (200 μM) than at the cmc.

Good conditions of measurement include saturation of enzyme. Concentrations used were as follows: (i) porcine pancreatic PLA₂: 1.5 mg/ml; and (ii) Crotalus durissus terrificus PLA₂: 0.43 mg/ml. The mother solution containing the inhibitor was prepared at an initial concentration of 10⁻² M. The IC₅₀ was determined using software coupled to an UV spectrophotometer. It calculated directly the initial velocity of the reaction. This velocity is represented by the following equation:

$${V}_{I} = \frac{Δ DO}{dt}$$

15 readings for each well (3 wells per concentration) were performed at intervals of 3 seconds.

### 11-2: Results

The results are given in Table 1 below in which the respective meanings of R, W, A, B, Y and D (Z and HET) in the formula (I) of the molecule tested are described in detail.

The results presented in Table 1 below demonstrate that the compounds of formula (I) tested have high selectivity against the PLA₂ of group II.

The compound Nos. 6-9 and 13-15, wherein p is 1 and Y is —CO—, possess the highest inhibitory activity. The compound Nos. 7, 8, 9, 12, 13 and 14 have high activity with a IC₅₀ value lower or equal to 0.3 μM against the human PLA₂ of group II.

### EXAMPLE 12

### In Vivo Activity Assay

### 12-1: Material and Methods

An in vivo activity assay was performed by a carrageenan-induced edema test in rats.

In the experimental protocol conducted, indomethacin as the reference product, or the compound No. 5, was administered intraperitoneally or orally at one hour before the injection of carrageenan into the hind leg of the rats. The volume of edema was measured at 0, 3 and 5 hours after the injection of carrageenan. The doses used were 5, 10 and 20 mg/kg for the two products tested.

### 12-2: Results

By the intraperitoneal route, the two products possessed an equivalent activity. Thus, at a dose of 10 mg/kg, the inhibitions of edema by indomethacin and the compound No. 5 were 79% and 73%, respectively.

By the oral route, the compound No. 5 had a higher activity than that of indomethancin, in which at 5 hours after the injection of carrageenan, the compound No. 5 inhibited 65% of edema but the reference product showed an inhibitory activity of 16%, when the two products were administered orally at a dose of 10 mg/kg.

### EXAMPLE 13

### Second In Vivo Activity Assay

Example 13 concerns an assay for the in vivo anti-inflammatory activity of certain compounds of the invention, by the ear edema test as an acute inflammatory experimental model.

A: Material and Methods

A-1: Material and Reagents

Six samples of compounds PMS 1227, PMS 1237, PMS 1281, PMS 1289, PMS 1314 and PMS 1315 were prepared.

The different compounds as listed above and their chemical identities are described in detail below.

PMS 1227

C₂₈H₄₆N₄O₂=470 g/mol

1-[4′-(4,5-dihydro-1,2,4(4H)-5-oxo-oxadiazol-3-ylmethyl)benzyl]-d-tetradecylpiperazine.

PMS 1281

C₂₈H₄₄N₄O₃+½H₂O=493 g/mol

1-[4′-(4,5-dihydro-1,2,4(4H)-5-oxo-oxadiazol-3-ylmethyl)benzyl]-4-tetradecylpiperazine.

PMS1289

C₂₈H₄₄N₄O₃=484 g/mol

1-(para((1,2,4-(4H)-5-oxo)oxodiazol-3-ylmethyl)benzoyl)-4-dodecyl-2,5-dimethylpiperazine.

PMS 1314

C₂₆H₄₀N₄O₃=456 g/mol

1-[4′-(4,5-dihydro-1,2,4(4H)-5-oxo-oxadiazol-3-ylmethyl)benzoyl]-4-dodecylpiperazine.

PMS 1315

C₂₉H₄₃N₃O₃S+1H₂O=531 g/mol

1-[4′-(2,4-dioxo-1,3-thiazolidine-5-ylidene)benzoyl]-4-tetradecylpiperazine.

As reagents, croton oil of special or premium grade, indomethacin (Sigma Co.), acetone, chloroform (100%), chloroform (80%), carboxymethylcellulose (CMC), ethanol, hexane, ether, polyethylene glycol (PEG) and saline solution were used.

A-2: As animals, male ICR mice weighing 25 g were used.

A-3: The following instruments were used: a gimlet for collecting skin samples, an apparatus for the measurement of ear thickness (Ozaki, Japan), a balance, an automatic pipette, pincettes, a Vortex stirrer, an anesthetic chamber, a hood, a cage, an Eppendorf® tube, a security cover, and tubes, etc.

A-4: To evaluate the in vivo anti-inflammatory effect of the inventive PMS compounds as listed above, the ear edema test was used as an acute inflammatory experimental model.

Measurement of Local Anti-Inflammatory Effect

After inducing edema on one ear of mice by the application of croton oil, the sample of each of the PMS compounds as described above was dissolved in 80% chloroform. The resulting solution was applied on the ear at the ratio of 1 mg of each compound per ear. The other ear was applied only with the solvent, i.e., 80% chloroform.

After 5 hours of the initiation of the experiment, the ear tissue at the level of edema was collected by a gimlet from the skin, and the tissue collected was compared with one collected from the control part of the ear by a gimlet, to calculate the percent inhibition.

Systemic Anti-Inflammatory Effect

The samples of the compounds as listed above were suspended in CMS, and administered orally at a ratio of 80 mg of each compound per mouse. At one hour after the initiation of the experiment, edema was induced by the application of croton oil.

At five hours after the application of croton oil, the tissues where edema had been developed were collected by a gimlet, and compared with the tissue collected from a control part, to calculate the percent inhibition.

A-5: To calculate the statistical significance, the results obtained in each of the control group and the control group were evaluated by Student's t-test.

B: Results

B-1: Local Anti-Inflammatory Effect

The local anti-inflammatory effect by topical administration of the PMS compound listed in the part of Materials and Methods in the ear edema test induced by croton oil is described in Table 2 below.

TABLE 2

| Test | Dose | Number of | Edema |
|---|---|---|---|
| compound | (mg/ear)/(mmol) | animals* | inhibition (%) |
| PMS 1227 | 1/0.00212 | 56 (7) | 51.45 ± 11.67 |
| PMS 1281 | 1/0.00206 | 56 (7) | 47.83 ± 12.34 |
| PMS 1289 | 1/0.00206 | 24 (3) | 62.79 ± 3.23 |
| PMS 1314 | 1/0.00219 | 48 (6) | 28.5 ± 6.85 |
| PMS 1315 | 1/0.00195 | 32 (4) | 32.12 ± 9.42 |
| Indomethacin | 0.5/0.00139 | 72 (9) | 43.11 ± 8.79 |

*The numbers in parenthesis imply the number of performed experiments.

According to a classification by the order of higher edema inhibition, the in vivo anti-inflammatory activities of the PMS compounds are as follows: PMS 1289>PMS 1227>PMS 1281>PMS 1315>PMS 1314.

B-2: Systemic Anti-Inflammatory Effect

The results obtained suggest that oral administration of the PMS compounds listed in the part of Materials and Methods did not induce a systemic anti-inflammatory effect in the oral edema experimental model induced by croton oil.

TABLE 1

| | 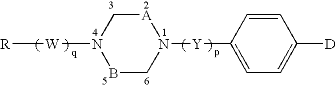 | | | | | | | | | |
|---|---|---|---|---|---|---|---|---|---|---|
| | | | | | D | | Anti-PLA₂ activity IC₅₀ (μM) | | | |
| Compound | R | A | B | Y | Z | HET | HPLA₂^{a} | PPLA₂^{b} | CB^{c} | |
| 1 2 | C₁₂H₂₅C₁₄H₂₉ | —CH₂— | —CH₂— | —CH₂— | —CH₂— | 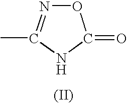 | >100 >10 | >100 >100 | Fluo | Spectro |
| Example 1 | | | | | | | | | | |
| 3 | C₁₆H₃₃ | | | | | | 10 | >100 | | |
| 4 | C₁₈H₃₇ | | | | | | 2.2 | >100 | | |
| 5 6 | C₁₄H₂₉C₁₈H₃₇ | —CH₂— | —CH₂— | C═O | —CH₂— | 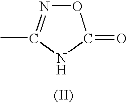 | 9 0.8 | >100 >100 | 3.8 1.5 | 2.6 2 |
| Example 2 | | | | | | | | | | |
| 7 | C₂₀H₄₁ | | | | | | 0.1 | >100 | | |
| 8 | C₂₂H₄₅ | | | | | | 0.1 | >100 | | |
| 9 | (C₉H₁₉)₂CH | | | | | | 0.3 | >100 | | |
| 10 Example 4 | C₁₈H₃₇ | —CH₂— | —CH₂— | Absent | —CH₂— | 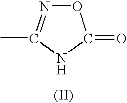 | 2.71 | >100 | | |
| 11 | C₁₈H₃₇ | CHMe | —CH₂— | C═O | —CH₂— | | 0.58 | >100 | | |
| 12 | C₁₈H₃₇ | CHMe | CHMe | C═O | —CH₂— | | 0.23 | >100 | Fluo | Spectro |
| 13 | C₁₂H₂₅ | CHMe | CHMe | C═O | —CH₂— | | 2.2 | >100 | | |
| Example 3 | | | | | | | | | | |
| 14 Example 5 | C₁₈H₃₇ | —CH₂— | —CH₂— | C═O | 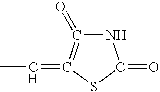 | | 0.28 | fluo 10 spectro >100 | 0.4 | 0.4 |
| 15 Example 6 | C₁₈H₃₇ | —CH₂— | —CH₂— | C═O | —CH₂— | 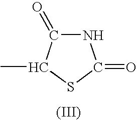 | 1 | >100 | 1.4 | 2 |

| | 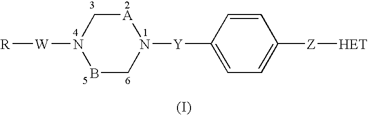 | | | | | | Anti-PLA₂ activity IC₅₀ (μM) | |
|---|---|---|---|---|---|---|---|---|
| Compound | R | A | B | Y | Z | HET | HPLA₂^{a} | PPLA₂^{b} |
| Example 7 | C₁₄H₂₉ | —CH₂— | —CO— | —CH₂— | —CH₂— | 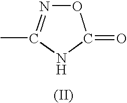 | 30 | >100 |
| Example 8 | C₁₄H₂₉ | —CH₂— | —CH₂— | C═O | —(CH₂)₂— | 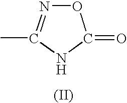 | 18.7 | >100 |
| Example 9 | C₁₄H₂₉ | —CH₂— | —CH₂— | C═O | —(CH₂)₃— | 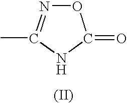 | 5.3 | >100 |

^{a}measured for fluorescence with human recombinant PLA₂ (group II)
^{b}measured for non-specific fluorescence with porcine pancreatic PLA₂ (group I)
^{c}measured with the PLA₂ of Crotalus durissus terrificus (group II) as indicated

1-10. (canceled)

11. A compound of formula (I): 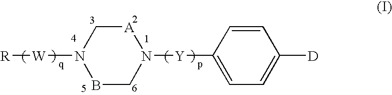
wherein:
D signifies a Z-HET group or a Z=HET group; and
(i) when D is a Z-HET group: HET is a five-membered heterocycle; and Z- is —(CR₁R₂)ₙ— or —(CR₁═CR₂)ₙ— where n is an integer from 1 to 6, and R₁ and R₂, which may be the same or different, independently is a hydrogen atom or a linear or branched alkyl group having 1 to 6 carbon atoms, and
(ii) when D is a Z=HET group, Z- together with the heterocycle represent a -Z=HET group of the following formula (IV) or (V) with the heterocycle: 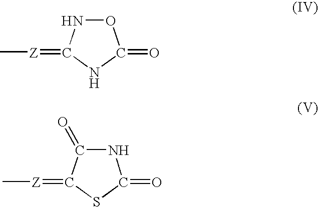
in which -Z= is —CR₁═ where R₁ is a hydrogen atom or a linear or branched alkyl group having 1 to 6 carbon atoms;
p is an integer of 0 or 1;
Y— is C═O, SO₂, or —(CR₃R₄)ₘ— where m is an integer from 1 to 6, and R₃ and R₄, which may be the same or different, independently are a hydrogen atom or a linear or branched alkyl group having 1 to 6 carbon atoms;
A and B, which may be the same or different, independently represent a carbon atom linked to hydrogen, or a carbon atom linked to both hydrogen and a linear or branched alkyl group having 1 to 3 carbon atoms, or a —C═O group;
q is an integer of 0 or 1;
W- is: 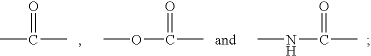
and
R is a linear or branched alkyl group having 1 to 22 carbon atoms, a polyaryl group, or an aryl-alkyl, alkyl-Q-alkyl, alkyl-Q-aryl, aryl-Q-aryl, aryl-Q-aryl, or aryl-Q-alkyl group where “aryl” is a substituted or unsubstituted 5- to 10-membered aryl group.

12. The compound of claim 11, wherein, when D signifies a Z-HET group: HET is an oxadiazolone of the following formula (II) or a thiazolidine dione of the following formula (III):

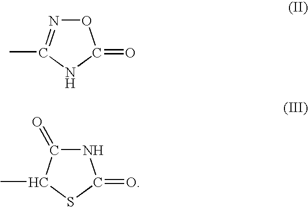

13. The compound of claim 11, wherein R is aryl-alkyl, alkyl-Q-aryl, aryl-Q-aryl or aryl-Q-alkyl wherein:
“aryl” is phenyl, naphthyl, phenylphenyl (or biphenyl) or heterocyclic aryl like an indolyl group; “alkyl” is a linear or branched alkyl group having 1 to 12 atoms; and “Q” is —O—, —S—, —NH—, —NR₅—, —NH—CO—NH—, 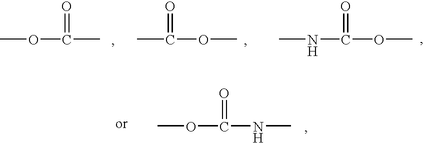
wherein R₅ is a linear or branched alkyl group having 1 to 6 carbon atoms.

14. The compound of claim 11, wherein p is equal to 1 and Y is a C═O group.

15. The compound of claim 11, further defined as:
a) 1-[4′-(4,5-dihydro-1,2,4(4H)-5-oxo-oxadiazol-3-ylmethyl)benzyl]-4-tetradecylpiperazine;
b) 1-[4′-(4,5-dihydro-1,2,4(4H)-5-oxo-oxadiazol-3-ylmethyl)benzoyl]-4-octadecylpiperazine;
c) 1-[4′-(4,5-dihydro-1,2,4(4H)-5-oxo-oxadiazole-3-ylmethyl)benzoyl]-2,5-dimethyl-4-dodecylpiperazine;
d) 1-[4′-(4,5-dihydro-1,2,4(4H)-5-oxo-oxadiazole-3-ylmethyl)phenyl]-4-octadecylpiperazine;
e) [4-(4′-octadecylpiperazine-1′-ylcarbonyl)benzylidene]-1,3-thiazolidine-2,4-dione;
f) 1-[4′-(2,4-dioxo-1,3-thiazolidine-5-ylmethyl)benzoyl]-4-octadecylpiperazine;
g) 1-[4′-(4,5-dihydro-1,2,4(4H)-5-oxo-oxadiazol-3-ylmethyl)benzyl]-4-tetradecylpiperazin-2-one;
h) 1-[4′-(4,5-dihydro-1,2,4(4H)-5-oxo-oxadiazol-3-ylethyl)benzoyl]-4-tetradecylpiperazine;
i) 1-[4′-(4,5-dihydro-1,2,4(4H)-5-oxo-oxadiazol-3-ylpropyl)benzoyl]-4-tetradecylpiperazine; or
j) 1-[4′-(4,5-dihydro-1,2,4(4H)-5-oxo-oxadiazol-3-yl-methylbenzoyl)-4-(N-octadecylaminocarbonyl)piperazine.

16. The compound of claim 11, further defined as comprised in a pharmaceutical composition.

17. A process for the preparation of a compound of formula (I): 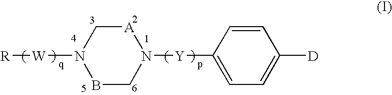
comprising:
a) reacting hydroxylamine hydrochloride with a derivative of the following formula (VI): 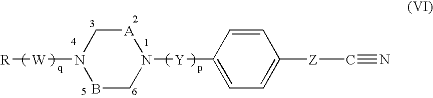
to form a corresponding intermediate oxime; and
b) subjecting the oxime to a cyclization reaction with chlorocarbonate (or chloroform) followed by heating to a temperature sufficient to achieve a practically complete cyclization:
wherein:
p is an integer of 0 or 1;
Y— is C═O, SO₂, or —(CR₃R₄)ₘ— where m is an integer from 1 to 6, and R₃ and R₄, which may be the same or different, independently are a hydrogen atom or a linear or branched alkyl group having 1 to 6 carbon atoms;
A and B, which may be the same or different, independently represent a carbon atom linked to hydrogen, or a carbon atom linked to both hydrogen and a linear or branched alkyl group having 1 to 3 carbon atoms, or a —C═O group;
q is an integer of 0 or 1;
W- is: 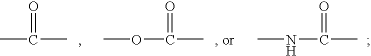
R is a linear or branched alkyl group having 1 to 22 carbon atoms, a polyaryl group, or an aryl-alkyl, alkyl-Q-alkyl, alkyl-Q-aryl, aryl-Q-aryl, aryl-Q-aryl, or aryl-Q-alkyl group where “aryl” is a substituted or unsubstituted 5- to 10-membered aryl group; and
Z is —(CR₁R₂)ₙ— or —(CR₁═CR₂)ₙ— where n is an integer from 1 to 6, and R₁ and R₂, which may be the same or different, independently are a hydrogen atom or a linear or branched alkyl group having 1 to 6 carbon atoms.

18. The process of claim 17, further comprising compounding the compound into a pharmaceutical composition.

19. A process for the preparation of a compound of formula (I): 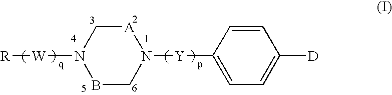
wherein:
D signifies a Z-HET group or a Z=HET group; and
(i) when D is a Z-HET group: HET is a five-membered heterocycle; and Z- is —(CR₁R₂)ₙ— or —(CR₁═CR₂)ₙ— where n is an integer from 1 to 6, and R₁ and R₂, which may be the same or different, independently is a hydrogen atom or a linear or branched alkyl group having 1 to 6 carbon atoms, and
(ii) when D is a Z=HET group, Z- together with the heterocycle represent a -Z=HET group of the following formula (IV) or (V) with the heterocycle: 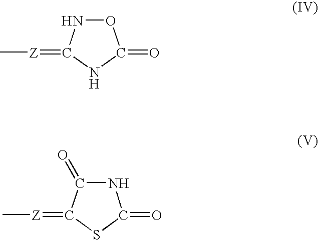
in which -Z= is —CR₁═ where R₁ is a hydrogen atom or a linear or branched alkyl group having 1 to 6 carbon atoms;
p is an integer of 0 or 1;
Y— is C═O, SO₂, or —(CR₃R₄)ₘ— where m is an integer from 1 to 6, and R₃ and R₄, which may be the same or different, independently are a hydrogen atom or a linear or branched alkyl group having 1 to 6 carbon atoms;
A and B, which may be the same or different, independently represent a carbon atom linked to hydrogen, or a carbon atom linked to both hydrogen and a linear or branched alkyl group having 1 to 3 carbon atoms, or a —C═O group;
q is an integer of 0 or 1;
W- is: 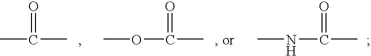
and
R is a linear or branched alkyl group having 1 to 22 carbon atoms, a polyaryl group, or an aryl-alkyl, alkyl-Q-alkyl, alkyl-Q-aryl, aryl-Q-aryl, aryl-Q-aryl, or aryl-Q-alkyl group where “aryl” is a substituted or unsubstituted 5- to 10-membered aryl group;
the process comprising reacting thiazolidine-2,4-dione with an aldehyde functional group of a derivative of formula (VII) to form the ethylene derivative of formula (V) 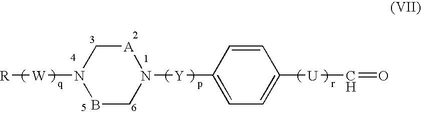
wherein:
r is an integer from 0 or 1; and
U is —(CR₆R₇)ₛ— or —(CR₆═CR₇)ₛ— where s is an integer from 1 to 6 and R₆ and R₇, which may be the same or different, independently are a hydrogen atom or a linear or branched alkyl group having 1 to 6 carbon atoms.

20. The process of claim 19, further comprising reducing the double linkage Z=C by catalytic hydrogenation.

21. The process of claim 19, further comprising compounding the compound into a pharmaceutical composition.

22. A method of preventing or treating inflammation comprising:
obtaining a compound of formula (I): 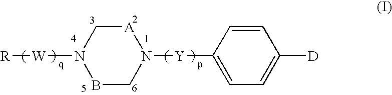
wherein:
D signifies a Z-HET group or a Z=HET group: and
(i) when D is a Z-HET group: HET is a five-membered heterocycle; and Z- is —(CR₁R₂)ₙ— or —(CR₁═CR₂)ₙ— where n is an integer from 1 to 6, and R₁ and R₂, which may be the same or different, independently is a hydrogen atom or a linear or branched alkyl group having 1 to 6 carbon atoms, and
(ii) when D is a Z=HET group, Z- together with the heterocycle represent a -Z=HET group of the following formula (IV) or (V) with the heterocycle: 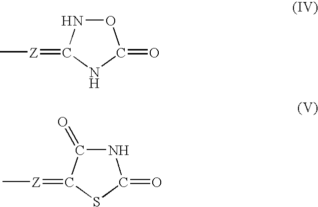
in which -Z= is —CR₁═ where R₁ is a hydrogen atom or a linear or branched alkyl group having 1 to 6 carbon atoms;
p is an integer of 0 or 1;
Y— is C═O, SO₂, or —(CR₃R₄)ₘ— where m is an integer from 1 to 6, and R₃ and R₄, which may be the same or different, independently are a hydrogen atom or a linear or branched alkyl group having 1 to 6 carbon atoms;
A and B, which may be the same or different, independently represent a carbon atom linked to hydrogen, or a carbon atom linked to both hydrogen and a linear or branched alkyl group having 1 to 3 carbon atoms, or a —C═O group;
q is an integer of 0 or 1;
W- is: 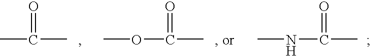
and
R is a linear or branched alkyl group having 1 to 22 carbon atoms, a polyaryl group, or an aryl-alkyl, alkyl-Q-alkyl, alkyl-Q-aryl, aryl-Q-aryl, aryl-Q-aryl, or aryl-Q-alkyl group where “aryl” is a substituted or unsubstituted 5- to 10-membered aryl group; and
administering the compound to a subject.
